(19) United States
(12) Patent Application Publication
(10) Pub. No.: US 2007/0207492 A1
(43) Pub. Date: Sep. 6, 2007

(54) Computational methods and systems to adjust a humoral immune response

(75) Inventors: Mahalaxmi Gita Bangera, Renton, WA (US); Muriel Y. Ishikawa, Livermore, CA (US); Edward K.Y. Jung, Bellevue, WA (US); Nathan P. Myhrvold, Medina, WA (US); Elizabeth A. Sweeney, Seattle, WA (US); Richa Wilson, San Francisco, CA (US); Lowell L. Wood JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/731,001

(22) Filed: Mar. 28, 2007

Related U.S. Application Data
(63) Continuation-in-part of application No. 10/925,902, filed on Aug. 24, 2004.

(51) Int. Cl. C12Q 1/68; G06F 19/00

(52) U.S. Cl. 435/6; 702/20

(57) ABSTRACT

The present application relates, in general, to a system and/or method related to detection and/or treatment.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

1. For purposes of the USPTO extra-statutory requirements, the present application constitutes a continuation in part of currently co-pending United States patent application entitled A SYSTEM AND METHOD RELATED TO ENHANCING AN IMMUNE SYSTEM naming MURIEL Y. ISHIKAWA, EDWARD K. Y. JUNG, NATHAN P. MYHRVOLD, RICHA WILSON, AND LOWELL L. WOOD, JR. as inventors, filed 24 Aug. 2004 having U.S. application Ser. No. 10/925,902.

2. For purposes of the USPTO extra-statutory requirements, the present application constitutes a continuation-in-part of currently co-pending United States parent application entitled A SYSTEM AND METHOD RELATED TO IMPROVING AN IMMUNE SYSTEM naming MURIEL Y. ISHIKAWA, EDWARD K. Y. JUNG, NATHAN P. MYHRVOLD, RICHA WILSON, and LOWELL L. WOOD, JR. as inventors, filed 24 Aug. 2004 having U.S. application Ser. No. 10/925,904.

3. For purposes of the USPTO extra-statutory requirements, the present application constitutes a continuation in part of currently co-pending United States patent application entitled A SYSTEM AND METHOD RELATED TO AUGMENTING AN IMMUNE SYSTEM naming MURIEL Y. ISHIKAWA, EDWARD K. Y. JUNG, NATHAN P. MYHRVOLD, RICHA WILSON, AND LOWELL L. WOOD, JR. as inventors, filed 24 Aug. 2004 having U.S. application Ser. No. 10/925,905.

4. For purposes of the USPTO extra-statutory requirements, the present application constitutes a continuation in part of currently co-pending United States patent application entitled A SYSTEM AND METHOD FOR MAGNIFYING AN IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 25 Aug. 2004 having U.S. application Ser. No. 10/926,753.

6. For purposes of the USPTO extra-statutory requirements, the present application constitutes a continuation in part of currently co-pending United States patent application entitled A SYSTEM AND METHOD FOR MODULATING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 01 Dec. 2004 having U.S. application Ser. No. 11/001,259.

7. For purposes of the USPTO extra-statutory requirements, the present application constitutes a continuation in part of currently co-pending United States patent application entitled A SYSTEM AND METHOD FOR HEIGHTENING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 03 Dec. 2004 having U.S. application Ser. No. 11,004,419.

8. For purposes of the USPTO extra-statutory requirements, the present application constitutes a continuation in part of currently co-pending United States patent application entitled A SYSTEM AND METHOD FOR AUGMENTING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 3 Dec. 2004 having U.S. application Ser. No. 11/004,446.

9. For purposes of the USPTO extra-statutory requirements, the present application constitutes a continuation in part of currently co-pending United States patent application entitled A SYSTEM AND METHOD FOR IMPROVING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 26 Jan. 2005 having U.S. application Ser. No. 11/044,656.

10. For purposes of the USPTO extra-statutory requirements, the present application constitutes a continuation in part of currently co-pending United States patent application entitled A SYSTEM AND METHOD FOR MAGNIFYING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 28 Jan. 2005 having U.S. application Ser. No. 11/046,658.

11. For purposes of the USPTO extra-statutory requirements, the present application constitutes a continuation in part of currently co-pending United States patent application entitled A SYSTEM AND METHOD FOR MAGNIFYING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 16 May, 2005 having U.S. application Ser. No. 11/131,155.

12. For purposes of the USPTO extra-statutory requirements, the present application constitutes a continuation in part of currently co-pending United States patent application entitled A SYSTEM AND METHOD FOR MODULATING A CELL MEDIATED IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 26 Aug. 2005 having U.S. application Ser. No. 11/213,325.

13. For purposes of the USPTO extra-statutory requirements, the present application constitutes a continuation in part of currently co-pending United States patent application entitled COMPUTATIONAL SYSTEMS AND METHODS RELATING TO FORTIFYING AN IMMUNE SYSTEM naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed 14 Mar. 2007 having U.S. application Ser. No. 11/724,593.

14. For purposes of the USPTO extra-statutory requirements, the present application constitutes a continuation in part of currently co-pending United States patent application entitled COMPUTATIONAL SYSTEMS AND METHODS RELATING TO AMELIORATING AN IMMUNE SYSTEM naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed 14 Mar. 2007 having U.S. application Ser. No. 11/724,580.

15. For purposes of the USPTO extra-statutory requirements, the present application constitutes a continuation in part of currently co-pending United States patent application entitled COMPUTATIONAL METHODS AND SYSTEMS TO REINFORCE A HUMORAL IMMUNE RESPONSE naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed 26 Mar. 2007 having U.S. application Ser. No. ______ [To Be Assigned by the USPTO].

16. For purposes of the USPTO extra-statutory requirements, the present application constitutes a continuation in part of currently co-pending United States patent application entitled COMPUTATIONAL METHODS AND SYSTEMS TO BOLSTER AN IMMUNE RESPONSE naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed contemporaneously herewith having U.S. application Ser. No. ______ [To Be Assigned by the USPTO].

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present application relates, in general, to detection and/or treatment.

### SUMMARY

In one aspect, a method includes but is not limited to: presenting one or more computable epitopes of at least one agent; predicting one or more pattern changes in the one or more computable epitopes of the at least one agent; and designating at least one immune response component operable for modulating (a) at least one of the one or more computable epitopes of the at least one agent or (b) at least one pattern-changed computable epitope. In addition to the foregoing, other method aspects are described in the claims, drawings, and text forming a part of the present application.

In one aspect, a system includes but is not limited to: circuitry for presenting one or more computable epitopes of at least one agent; circuitry for predicting one or more pattern changes in the one or more computable epitopes of the at least one agent; and circuitry for designating at least one immune response component operable for modulating (a) at least one of the one or more computable epitopes of the at least one agent or (b) at least one pattern-changed computable epitope. In addition to the foregoing, other system aspects are described in the claims, drawings, and text forming a part of the present application.

In one aspect, a system includes but is not limited to: a computer readable medium including, but not limited to, a computer program for use with a computer system and wherein the computer program includes a plurality of instructions including one or more instructions for presenting one or more computable epitopes of at least one agent; one or more instructions for predicting one or more pattern changes in the one or more computable epitopes of the at least one agent; and one or more instructions for designating at least one immune response component operable for modulating at least one pattern-changed computable epitope. In addition to the foregoing, other system aspects are described in the claims, drawings, and text forming a part of the present application.

In one aspect, a program product includes but is not limited to: at least one signal bearing medium including one or more instructions for presenting one or more computable epitopes of at least one agent; one or more instructions for predicting one or more pattern changes in the one or more computable epitopes of the at least one agent; and one or more instructions for designating at least one immune response component operable for modulating at least one pattern-changed computable epitope. In addition to the foregoing, other program product aspects are described in the claims, drawings, and text forming a part of the present application.

In one aspect, a method includes but is not limited to: presenting one or more antigens of at least one agent; predicting one or more pattern changes in the one or more antigens of the at least one agent; and designating at least one immune response component operable for modulating at least one pattern-changed antigen. In addition to the foregoing, other method aspects are described in the claims, drawings, and text forming a part of the present application.

In one aspect, a system includes but is not limited to: circuitry for presenting one or more antigens of at least one agent; circuitry for predicting one or more pattern changes in the one or more antigens of the at least one agent; and circuitry for designating at least one immune response component operable for modulating at least one pattern-changed antigen. In addition to the foregoing, other system aspects are described in the claims, drawings, and text forming a part of the present application.

In one aspect, a system includes but is not limited to: a computer readable medium including, but not limited to, a computer program for use with a computer system and wherein the computer program includes a plurality of instructions including: one or more instructions for presenting one or more antigens of at least one agent; one or more instructions for predicting one or more pattern changes in the one or more antigens of the at least one agent; and one or more instructions for designating at least one immune response component operable for modulating at least one pattern-changed antigen. In addition to the foregoing, other system aspects are described in the claims, drawings, and text forming a part of the present application.

In one aspect, a system related to an immune response includes but is not limited to: circuitry for predicting one or more pattern changes in one or more antigens of at least one agent; and circuitry for designating at least one immune response component operable for modulating at least one pattern-changed antigen. In addition to the foregoing, other system aspects are described in the claims, drawings, and text forming a part of the present application.

In one aspect, a method includes but is not limited to: presenting one or more epitopes of at least one agent; predicting one or more pattern changes in the one or more epitopes of the at least one agent; and designating at least one immune response component operable for modulating at least one pattern-changed epitope. In addition to the foregoing, other method aspects are described in the claims, drawings, and text forming a part of the present application.

In one aspect, a system includes but is not limited to: circuitry for presenting one or more epitopes of at least one agent; circuitry for predicting one or more pattern changes in the one or more epitopes of the at least one agent; and circuitry for designating at least one immune response component operable for modulating at least one pattern-changed epitope. In addition to the foregoing, other system aspects are described in the claims, drawings, and text forming a part of the present application.

In one aspect, a system includes but is not limited to: a computer readable medium including, but not limited to, a computer program for use with a computer system and wherein the computer program includes a plurality of instructions including one or more instructions for presenting one or more epitopes of at least one agent, one or more instructions for predicting one or more pattern changes in the one or more epitopes of the at least one agent, and one or more instructions for designating at least one immune response component operable for modulating at least one pattern-changed epitope. In addition to the foregoing, other system aspects are described in the claims, drawings, and text forming a part of the present application.

In one aspect, a program product includes but is not limited to: at least one signal bearing medium including one or more instructions for presenting one or more epitopes of at least one agent, one or more instructions for predicting one or more pattern changes in the one or more epitopes of the at least one agent, and one or more instructions for designating at least one immune response component operable for modulating at least one pattern-changed epitope. In addition to the foregoing, other program product aspects are described in the claims, drawings, and text forming a part of the present application.

In one aspect, a method related to an immune response includes but is not limited to: specifying an agent; and presenting one or more epitopes of the specified agent. In addition to the foregoing, other method aspects are described in the claims, drawings, and text forming a part of the present application.

In one aspect, a system related to an immune response includes but is not limited to: circuitry for specifying an agent; and circuitry for presenting one or more epitopes of the specified agent. In addition to the foregoing, other system aspects are described in the claims, drawings, and text forming a part of the present application.

In one or mote various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In addition to the foregoing, various other method and or system aspects are set forth and described in the text (e.g., claims and/or detailed description) and/or drawings of the present application.

The foregoing is a summary and thus contains, by necessity; simplifications, generalizations and omissions of detail; consequently, those skilled in the art will appreciate that the summary is illustrative only and is not intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE FIGURES

FIG. 1 depicts one aspect of a system that may serve as an illustrative environment of and/or for subject matter technologies.

FIG. 2 depicts a partial view of a system that may serve as an illustrative environment of and/or for subject matter technologies.

FIG. 3 depicts a partial view of a system that may serve as an illustrative environment of and/or for subject matter technologies.

FIG. 4 depicts a diagrammatic view of one aspect of an exemplary interaction of an immune response component, for example, an antibody interacting with an epitope displayed by an agent.

FIG. 5 depicts a diagrammatic view of one aspect of a method of enhancing an immune response.

FIG. 6 depicts one aspect of an antigen-antibody interaction showing the occurrence of mutational changes in a selected epitope and corresponding changes in a complementary antibody.

FIG. 7 is an illustration of one aspect of mutational changes in an epitope displayed by an agent and the corresponding changes in an immune response component, for example, an antibody.

FIG. 8 depicts a high-level logic flow chart of a process.

FIG. 9 depicts a high-level logic flowchart depicting alternate implementations of the high-level logic flowchart of FIG. 8.

FIG. 10 depicts a high-level logic flowchart depicting alternate implementations of the high-level logic flowchart of FIG. 8.

FIG. 11 depicts a high-level logic flowchart depicting alternate implementations of the high-level logic flowchart of FIG. 8.

FIG. 12 depicts a high-level logic flowchart depicting alternate implementations of the high-level logic flowchart of FIG. 8.

FIG. 13 depicts a high-level logic flowchart depicting alternate implementations of the high-level logic flowchart of FIG. 8.

FIG. 14 depicts a high-level logic flowchart depicting alternate implementations of the high-level logic flowchart of FIG. 8.

FIG. 15 depicts a high-level logic flowchart depicting alternate implementations of the high-level logic flowchart of FIG. 8.

FIG. 16 depicts a high-level logic flowchart depicting alternate implementations of the high-level logic flowchart of FIG. 8.

FIG. 17 depicts a high-level logic flowchart depicting alternate implementations of the high-level logic flowchart of FIG. 8.

FIG. 18 depicts a high-level logic flowchart depicting alternate implementations of the high-level logic flowchart of FIG. 8.

The use of the same symbols in different drawings typically indicates similar or identical items.

### DETAILED DESCRIPTION

The present application uses formal outline headings for clarity of presentation. However, it is to be understood that the outline headings are for presentation purposes, and that different types of subject matter may be discussed throughout the application (e.g., device(s)/structure(s) may be described under the process(es)/operations heading(s) and/or process(es)/operations may be discussed under structure(s)/process(es) headings). Hence, the use of the formal outline headings is not intended to be in any way limiting.

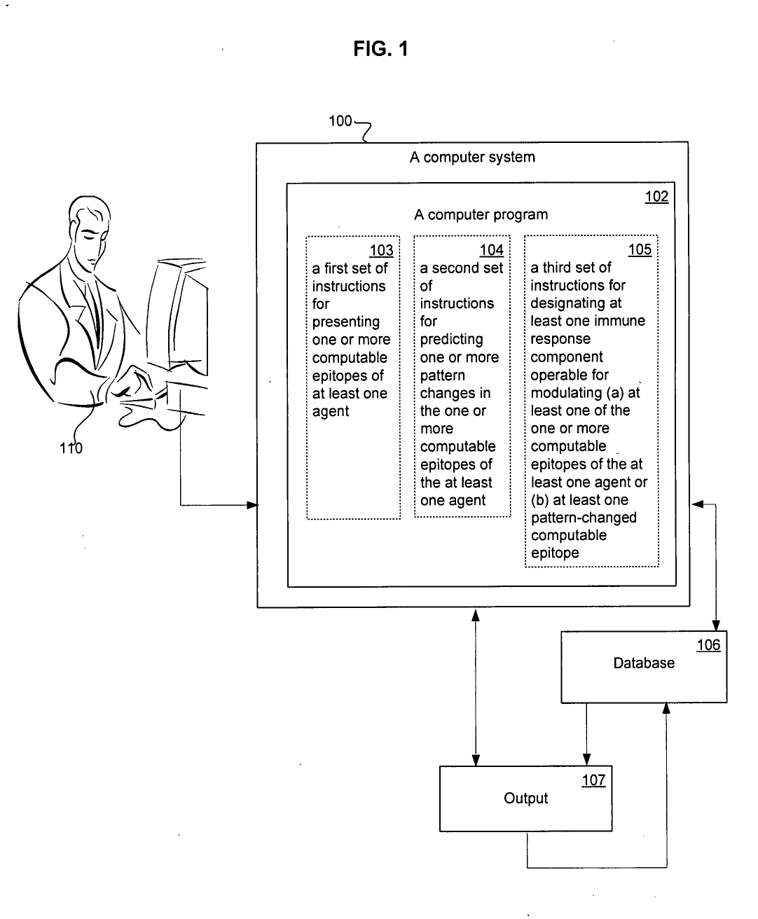

With reference now to the Figures and with reference now to FIG. 1, depicted is one aspect of a system that may serve as an illustrative environment of and/or for subject matter technologies, for example, a computer-based method for designating an immune response component for modulating an epitope and/or a computable epitope displayed by an agent. Accordingly, the present application first describes certain specific exemplary systems of FIG. 1; thereafter, the present application illustrates certain specific exemplary structures and processes. Those having skill in the art will appreciate that the specific structures and processes described herein are intended as merely illustrative of their more general counterparts. It will also be appreciated by those of skill in the art that an epitope-antibody, a computable epitope-antibody interaction, an immune cell receptor-epitope and/or immune-cell secretion product-epitope, and/or an antigen-antibody interaction is an exemplary interaction of an immune response component with an epitope, a computable epitope, and/or an antigen. Therefore, although, the exact nature of the interaction may vary, the overall picture as described herein and/or in other related applications relates to the interaction of an immune response component interacting with the epitope, computable epitope, and/or the antigen. As used herein, the term “epitope” may, if appropriate to context, be used interchangeably with computable epitope, antigen, paratope binding site, antigenic determinant, and/or determinant.

A. Structure(s) and or System(s)

Continuing to refer to FIG. 1, depicted is a partial view of a system that may serve as an illustrative environment of and/or for subject matter technologies. One or more users 110 may use a computer system 100 including a computer program 102, for example, for identifying epitopes associated with a disease, disorder, or condition. The computer program 102 may include one or more sets of instructions, for example, a first set of instructions 103 for presenting one or more computable epitopes of at least one agent, for example, may designate the selection of at least one computable epitope based on some parameters. The computer program 102 may include a second set of instructions 104 for predicting one or more pattern changes in the one or more computable epitopes of the at least one agent, for example, mutations, variations or alternate computable epitopes. The computer program 102 may include a third set of instructions 105 for designating at least one immune response component operable for modulating (a) at least one of the one or more computable epitopes of the at least one agent or (b) at least one pattern-changed computable epitope, for example, including, but not limited to, a natural and/or a synthetic antibody. The computer program 102 may accept input, for example, from medical personnel, a researcher, or wet lab personnel. A user interface may be coupled to provide access to the computer program 102. In one implementation, the computer program 102 may access a database 106 storing information and transmit an output 107 to the computer system 100. In one exemplary implementation a feedback loop is set up between the computer program 102 and the database 106. The output 107 may be fed back into the computer program 102 and/or displayed on the computer system 100. The system may be used as a research tool, as a tool for furthering treatment or the like. This feedback scheme may be useful in an iterative process such as described herein and elsewhere.

In various aspects, the computer system 100, the computer program 102 and/or the circuitry include predictive algorithms for determining the pattern changes in the computable epitope and the sequence of the computable epitope. In other various aspects, the computer system 100, the computer program 102 and/or the circuitry include predictive algorithms for determining the course of a disease influenced by the pattern changes in the computable epitope of the agent. In various aspects, the computer system 100, the computer program 102 and/or the circuitry includes computer-based modeling software for designing and selecting the immune response component for reducing the ability of the agent to establish itself in a host and/or to cause a disease, disorder and/or a condition that requires management. In other various aspects, the computer system 100, the computer program 102 and/or the circuitry includes software for integrating with other computer-based systems and incorporating information relevant to selecting an immune response component for modulating the computable epitopes.

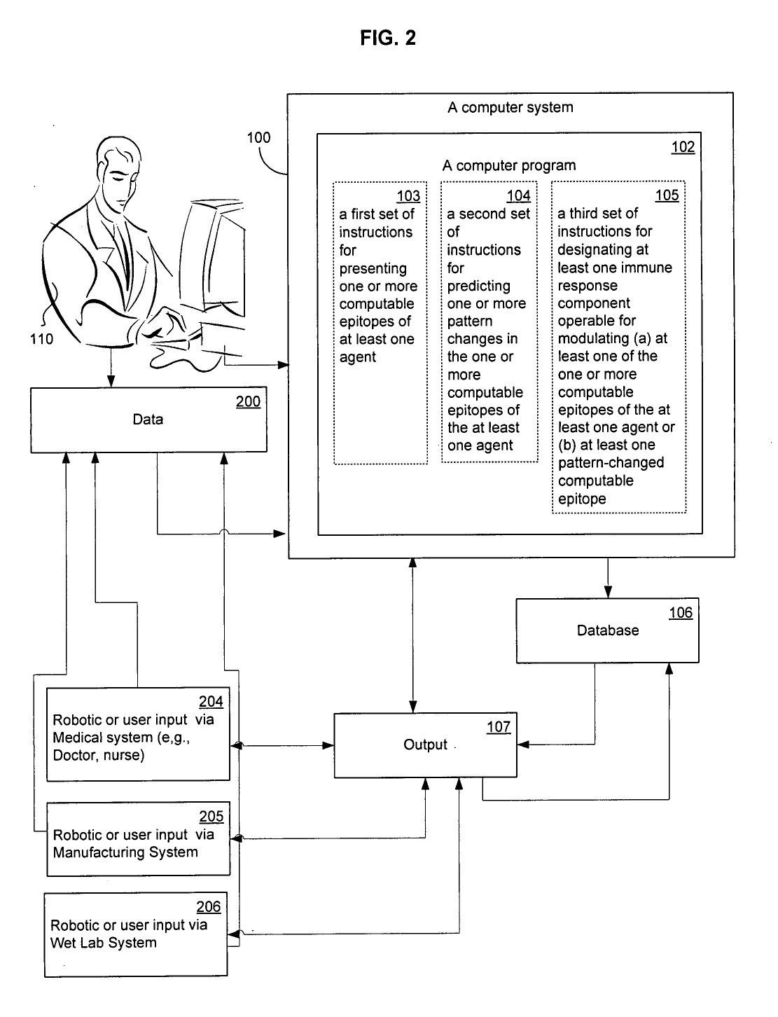

With reference to the figures, and with reference now to FIG. 2, depicted is a partial view of a system that may serve as an illustrative environment of and/or for subject matter technologies. The database 106, data 200, and/or the output 107 may be accessed by various input mechanisms, for example, mechanisms including but not limited to, robotic and/or user input via a medical system 204, robotic and/or user input via manufacturing system 205, or robotic and/or user input via wet lab system 206. Access to the data 200 may be provided, for example, for further manipulation of the data.

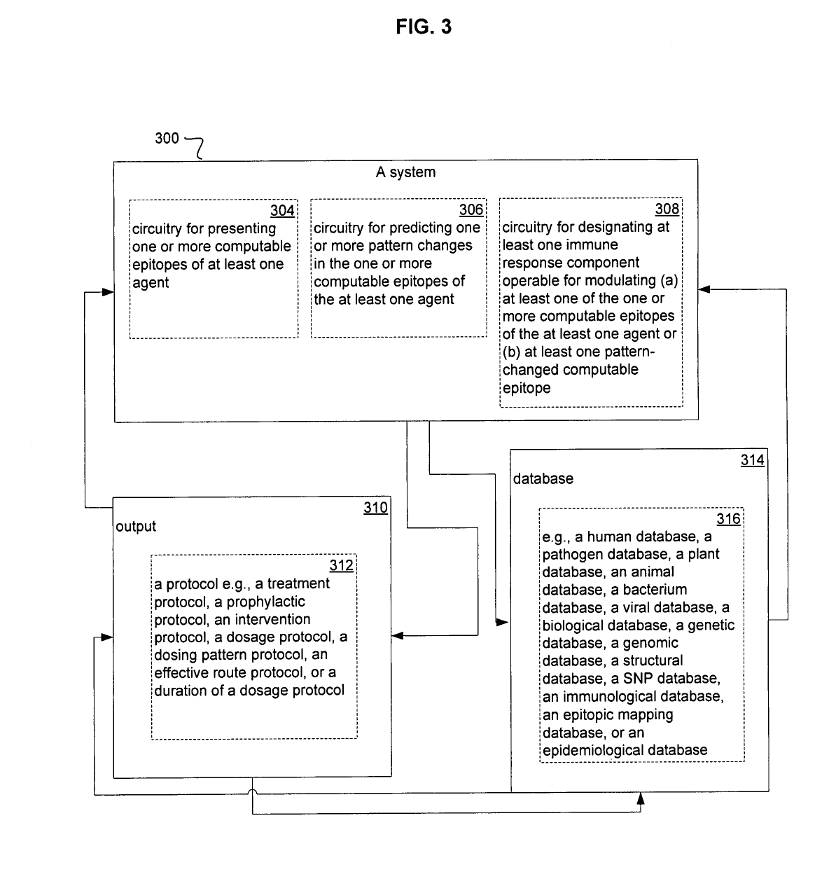

With reference to the figures, and with reference now to FIG. 3, depicted is a partial view of a system that may serve as an illustrative environment of and/or for subject matter technologies. In one aspect, a system 300 may include circuitry and/or components 304 for presenting one or more computable epitopes of at least one agent, circuitry and/or components 306 for predicting one or more pattern changes in the one or more computable epitopes of the at least one agent, and circuitry and/or components 308 for designating the at least one immune response component operable for modulating (a) at least one of the one or more computable epitopes of the at least one agent or (b) at least one pattern-changed computable epitope. Those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure.

Continuing to refer to FIG. 3, the system 300 may be coupled to a database 314 of an identifiable type 316, for example, including, but not limited to, a human database, a pathogen database, a plant database, an animal database, a bacterium database, a viral database, a biological database, a genetic database, a genomic database, a structural database, a SNP database, an immunological database, an epitopic mapping database, and/or an epidemiological database. An output 310 may be displayed, for example, in the form of a protocol 312, for example, including but not limited to a treatment protocol, a prophylacetic protocol, a therapeutic protocol, an intervention protocol, a dosage protocol, a dosing pattern (in space, in time or in some combination thereof) protocol, an effective route protocol, and/or a duration of a dosage protocol. In one aspect the type of output 310 may be selected by the user.

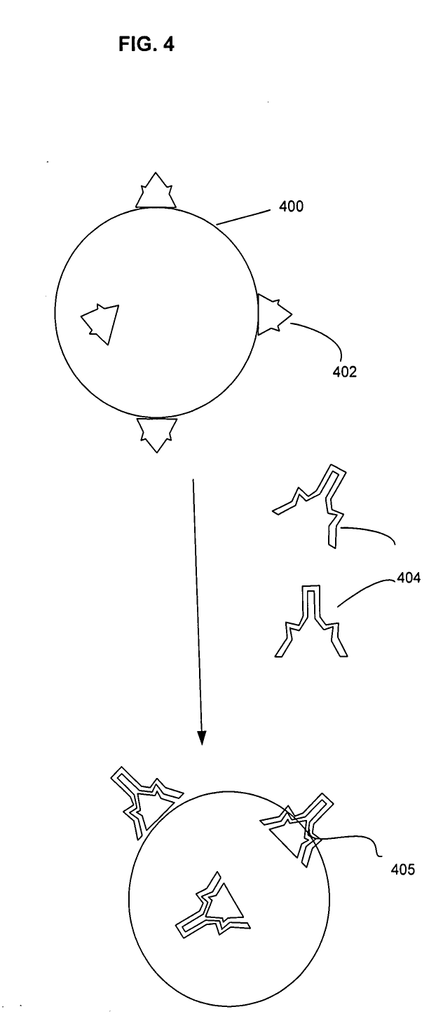

With reference to the figures, and with reference now to FIG. 4, depicted is a diagrammatic view of one aspect of an exemplary interaction of an immune response component, for example, an antibody 404 interacting with an epitope 402 displayed by an agent 400, for example, including but not limited to, in consequence of an interaction involving the agent 400.

The term “immune response component,” as used herein, may include, but is not limited to, at least a part of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a CD1 molecule, a B lymphocyte, an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, an antibody fragment, and/or a synthetic antibody and/or any component of the immune system that may bind to an antigen and/or an epitope thereof in a specific and/or a useful manner.

The term “agent”, as used herein, may include, for example, but is not limited to, an organism, a virus, a bacterium, a mycobacterium, a phage, a yeast, a mold, a fungus, a mycoplasma, an ureaplasma, a chlamydia, a rickettsial organism, a protoctist, an archaeal organism, a nanobacterium, a prion, an agent responsible for a transmissible spongiform encephalopathy (TSE), a multicellular parasite, a protein, an infectious protein, a nucleic acid, an infectious nucleic acid, a polymeric nucleic acid, a metabolic byproduct, a cellular byproduct, and/or a toxin. The term “agent” 400 may include, but is not limited to, a putative causative agent of a disease or disorder, or a cell or component thereof that is deemed, for example, a target for therapy, a target for neutralization, and/or or a cell whose removal, lysis or functional degradation may prove beneficial to the host. The term “agent” 400 may also include, but is not limited to, a byproduct or output of a cell that may be neutralized and/or whose removal or functional neutralization may prove beneficial to the host. Furthermore, the term “agent” may include an agent belonging to the same family or group as the agent of primary interest, or an agent exhibiting a common and/or a biological function relative to the agent of primary interest.

The term “antibody” as used herein, is used in the broadest possible sense and may include but is not limited to an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. The term “antibody” may also include but is not limited to types of antibodies such as IgA, IgD, IgE, IgG and/or IgM, and/or the subtypes IgG1, IgG2, IgG3, IgG4, IgA1 and/or IgA2. The term “antibody” may also include but is not limited to an antibody fragment such as at least a portion of an intact antibody for instance, the antigen-binding variable region. Examples of antibody fragments include Fv, Fab, Fab′, F(ab′), F(ab′).sub.2, Fv fragment, diabody, linear antibody, single-chain antibody molecule, multispecific antibody, and/or other antigen-binding sequences of an antibody. Additional information may be found in: U.S. Pat. No. 5,641,870; U.S. Pat. No. 4,816,567; WO 93/11161; Holliger et al., Diabodies: small bivalent and bispecific antibody fragments, PNAS, 90: 6444-6448 (1993); and Zapata et al., Engineering linear F(ab′)2 fragments for efficient production in Escherichia coli and enhanced antiproliferative activity, Protein Eng. 8(10): 1057-1062 (1995), which are incorporated herein by reference. Antibodies may be generated for therapeutic purposes by a variety of known techniques, such as, for example, phage display, and/or transgenic animals.

The term “antibody”, as used herein, may include anti-idiotypic antibodies. Anti-idiotypic antibodies may elicit a stronger immune response compared to the antigen and may be used for enhancing the immune response. Anti-idiotypic antibodies may be rapidly selected, for example, by phage display technology. Additional information may be found in U.S. Patent Application No. 20040143101, to Soltis, which is incorporated herein by reference. The term “antibody”, as used herein, also may include, but is not limited to, functional derivatives of a monoclonal antibody, which include antibody molecules or fragments thereof that have retained a dominant fraction of the antigenic specificity and the functional activity of the parent molecule.

The term “heteroantibody,” as used herein, may include but is not limited to, two or more antibodies, antibody fragments, antibody derivatives, and/or antibodies with at least one specificity that are linked together. Additional information may be found in U.S. Pat. No. 6,071,517, which is incorporated herein by reference.

The term “chimeric antibody,” as used herein, may include but is not limited to antibodies having mouse-variable regions joined to human-constant regions. In one aspect, “chimeric antibody” includes antibodies with human framework regions combined with complementarity-determining regions (CDRs) obtained from a mouse and/or rat; however those skilled in the art will appreciate that CDRs may be obtained from other sources. Additional information may be found in EPO Publication No 0239400, which is incorporated herein by reference.

The term “humanized antibody,” as used herein, may include but is not limited to an antibody having one or more human-derived regions, and/or a chimeric antibody with one or more human-derived regions, also considered the recipient antibody, combined with CDRs from a donor mouse and/or rat immunoglobulin. In one aspect, a humanized antibody may include residues not found in either donor and/or recipient sequences. A humanized antibody may have single and/or multiple specificities. Additional information may be found in U.S. Pat. No. 5,530,101, and U.S. Pat. No. 4,816,567, which are incorporated herein by reference. Information may also be found in: Jones et al., Replacing the complementarity-determining regions in a human antibody with those from a mouse, Nature, 321:522-525(1986); Riechmann et al., Reshaping human antibodies for therapy, Nature, 332:323-327 (1988); and Verhoeyen et al., Reshaping human antibodies: grafting an antilysozyme activity, Science, 239:1534 (1988), which are all incorporated herein by reference.

The term “human antibody,” as used herein, may include but is not limited to an antibody with variable and constant regions derived from human germline immunoglobulin sequences. The term “human antibody” may include, and is not limited to, amino acid residues of non-human origin, encoded by non-human germline, such as, for example, residues introduced by site-directed mutations, random mutations, and/or insertions. Methods for producing human antibodies are known in the art and incorporated herein by reference. Additional information may be found in U.S. Pat. No. 4,634,666, which is incorporated herein by reference.

The term “recombinant antibody,” as used herein, may include antibodies formed and/or created by recombinant technology, including, but not limited to, chimeric, human, humanized, hetero antibodies and/or the like.

The term “epitope”, as used herein, may include, but is not limited to, a sequence of at least 3 amino acids, a sequence of at least nine nucleotides, an amino acid, a nucleotide, a carbohydrate, a protein, a lipid, a capsid protein, a polysaccharide, a lipopolysaccharide, a glycolipid, a glycoprotein, and/or or at least a part of a cell. As used herein, the term “epitope” may, if appropriate to context, be used interchangeably with antigen, paratope binding site, antigenic determinant, and/or determinant. As used herein, the term “determinant” can include an influencing element, determining element, and/or factor, unless context indicates otherwise. In one aspect, the term “epitope” includes, but is not limited to, a peptide-binding site. As used herein, the term “epitope” may include structural and/or functionally similar sequences found in the agent. The term “epitope” includes, but is not limited to, similar sequences observed in orthologs, paralogs, homologs, isofunctional homologs, heterofunctional homologs, heterospecific homologs, and/or pseudogenes of the agent. The epitope may include any portion of the agent. In one aspect, the epitope may include at least a portion of a gene or gene-expression product. In another aspect, the epitope may include at least a part of a non-coding region.

The term “computable epitope” as used herein, includes, but is not limited to, an epitope whose likely future mutable forms may be predicted by using, for example, including, but not limited to, practicable computer based predictive methodology and/or practicable evolutionary methods and/or practicable probabilistic evolutionary models and/or practicable probabilistic defect models and/or practicable probabilistic mutation models. For example, Smith et al. in their article “Mapping the Antigenic and Genetic Evolution of Influenza Virus” on the history of the antigenic evolution of the human influenza virus, Science 305, 371 (2004), which is incorporated herein by reference in its entirety, present in this paper's Table 1 and the supporting text thereof a set of patterns of viral coat-protein epitopic evolution which constitutes a basis for predicting one or more patterns of epitopic evolution in this particular agent, which is a well-established threat to human physiological well-being. In one aspect, the computable epitope may be suggested by, for example, including but not limited to, predictive parallel extrapolations with similar structure, key residues, and/or the presence or absence of known domains. In another aspect, mathematics, statistical analysis and/or biological structural modeling tools may provide the relevant information for designating or identifying the computable epitope. One specific example of a computable epitope is a polypeptide associated with the HIV-1 virus, which may be, for example, seven to ten amino acids long. Knowing any starting state of such a polypeptide (e.g., how the various amino acids are sequenced/arranged), and using current computational techniques, it is practicable to calculate the likely future combinations of the seven to ten amino acids in the peptide so as to be able to predict how the epitope will likely appear as evolution/change occurs in the epitope as biological processes progress. Indeed, many such evolutionary progressions in the protein sequences of the viral proteins (e.g., reverse transcriptase and protease) of the several major strains of HIV-1 virus have been reported in the literature, and are used for monitoring the clinical progression of disease in patients. Consequently, in some implementations, technologies described herein computationally predict how the epitope(s) will appear in future mutable forms. This predictive knowledge allows for the designation of at least one immune response component operable for modulating (e.g., reducing and/or eliminating) at least one “future version” of some posited presently existing epitope. As a specific example, one might predict the five or six mostly likely ways in which at least one epitope of a viral protein of a current strain of HIV-1 might appear a few months in the future, and then designate that a person's immune cells be exposed to the chemical structures of the epitopes of such an essential protein of such future HIV-1 strains to produce an immune response ready, waiting, and keyed to such future epitopic variants of the at least one HIV-1 strain. Once such antibodies or other immune response components have been produced, amplification or adjuvant techniques may be utilized to produce usefully-large quantities of such antibodies or other immune responses at a time earlier than the elapsing of the three months, and such antibodies administered to a host, or a vaccine eliciting such antibodies administered to a host, or cytotoxic responses prepared in the host, and/or a combination thereof. Then, if the HIV-1 virus does evolve or mutate in at least one of the five or six computationally-predicted ways, antibodies or other specific immune responses will be present and waiting to “lock onto” and negate the HIV-1 virus as it mutates along the predicted paths, thereby effectively precluding its ‘mutational escape’ from the initial therapy. Examples listed supra are merely illustrative of methodology that may be used for designating the computable epitope and are not intended to be in any way limiting.

Continuing to refer to FIG. 4, the epitope 402 or parts thereof may be displayed by the agent 400, may be displayed on the surface of the agent 400, extend from the surface of the agent 400, and/or may only be partially accessible by the immune response component. In one aspect, the epitope 402 may be a linear determinant. For example, the sequences may be adjacent to each other. In another aspect, the epitope 402 is a non-linear determinant, for example, including juxtaposed groups which are non-adjacent ab initio but become adjacent to each other on folding or other assembly. Furthermore, the sequence of the non-linear determinant may be derived by proteasomal processing and/or other mechanisms (e.g., glycosylation, or the superficial ‘decoration’ of proteins with sugars) and the sequence synthetically prepared for presentation to the immune response component.

Continuing to refer to FIG. 4, in one aspect, the immune system launches a humoral response producing antibodies capable of recognizing and/or binding to the epitope 402 followed by the subsequent lysis of the agent 400. Mechanisms by which the epitope 402 elicits an immune response are known in the art and such mechanisms are incorporated herein by reference. In one aspect, the binding of the antibody 404 to the epitope 402 to form an antigen-antibody complex 405 is characterized as a lock-and-key fit. In another aspect, the binding affinity of the antibody for the epitope may vary in time (e.g., in the course of ‘affinity maturation’) or with physiological circumstances. In yet another aspect, the epitope-antibody complex may bind with varying degrees of reversibility. The binding or the detachment of the epitope-antibody complex may be manipulated, for example, by providing a small (possibly solvated) atom, ion, molecule or compound that promotes the association or disassociation.

In one aspect, the epitope 402 is capable of evoking an immune response. The strength and/or type of the immune response may vary, for example, the epitope 402 may invoke a weak response and/or a medium response as measured by the strength of the immune response. It is contemplated that in one instance the epitope 402 selected for targeting may be one that invokes a weak response in the host; however, it may be selective to the agent 400. In another example, the epitope 402 selected may invoke a weak response in the host; however, it may be selected for targeting as it is common to a number of agents deemed as targets. The herein described implementations are merely exemplary and should be considered illustrative of like and/or more general implementations within the ambit of those having skill in the art in light of the teachings herein.

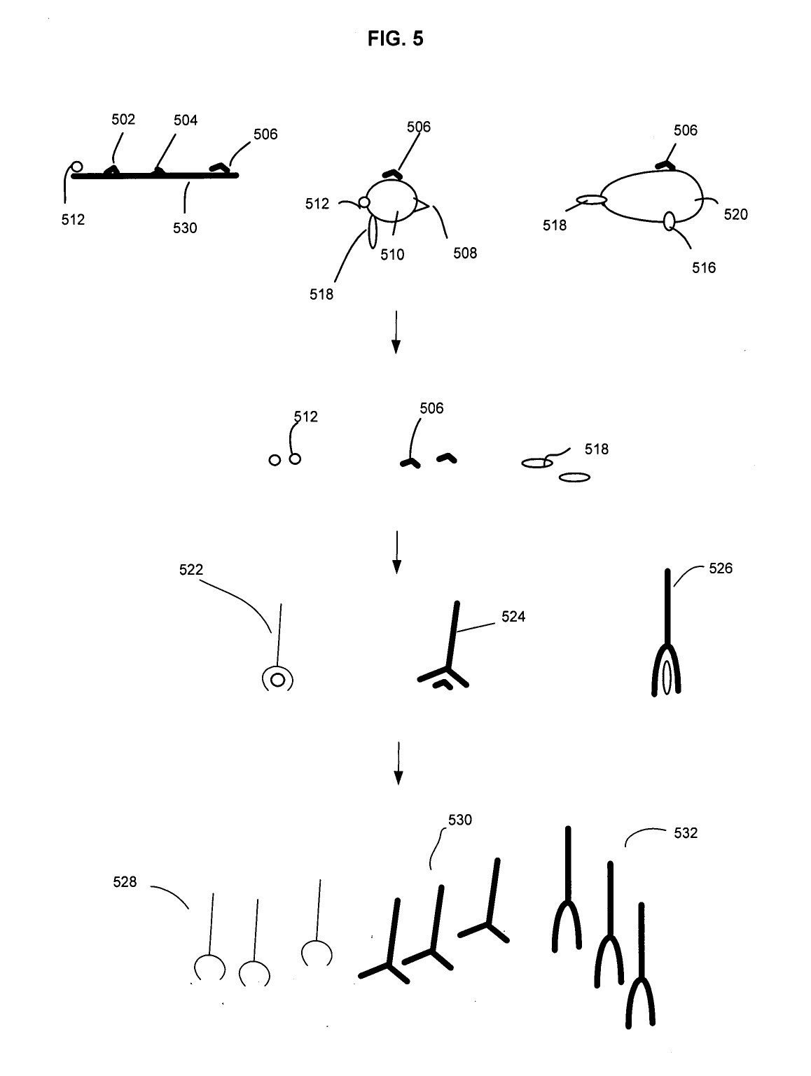

With reference to the figures, and with reference now to FIG. 5, depicted is a diagrammatic view of one aspect of a method of enhancing an immune response. In one aspect, an effective treatment therapy towards a disease and/or a disorder may utilize one or more immune response components designed to recognize one or more epitopes common to one or more agents. Such common or shared epitopes may represent an effective target group of epitopes. The immune response components designed to seek out and neutralize the common epitopes may be effective against one or more agents.

In one aspect, the one or more agents may be subtypes of the agent 400. In this aspect, a set of epitopes may be selected for targeting an agent. In another aspect, the one or more agents may be opportunistic agents capable of aiding or exaggerating an infection formed by the agent 400. In yet another aspect, the one or more agents may be agents known to establish a foothold in the host organism prior to or subsequent to an infection or in response to a person's lowered immune response.

With reference now to FIGS. 4 and 5, in one aspect, a shared epitope 506 is depicted as common to three agents 530, 510 and 520. In another aspect, a second shared epitope 512 is common to two agents 530 and 510. In yet another aspect, a third shared epitope 518 is common to two agents 510 and 520. However, not all epitopes are shared epitopes. For example, in FIG. 5, epitopes 502 and 504 of agent 530 are not shared by agents 510 or 520. Finding a subset of common epitopes shared amongst one or more agents may be done by statistical analysis, for example, by metaprofiling.

Continuing to refer to FIGS. 4 and 5, in one aspect, one or more agents 530, 510, and 520 depicted may share a subset of common epitopes. The selection of epitopes may depend on a number of criteria. For example, the initial selection may be based on selection criteria including, but not limited to, the number of instances of presentation of the epitope 402 by one or more agents, the number of instances of presentation of the epitope 402 by the agent 400, the location of the epitope 402, the size of the epitope 402, the nature of the epitope 402, the comparative sequence identity and/or homology of the epitope 402 with host sequences, the composition of the epitope 402, and/or putative known or predicted changes in the epitope 402 sequence. The selection of epitopes may also depend on, for example, the type of immune response component desired for treating and/or managing the disease, disorder, and/or condition.

In one aspect, the epitope 402 selected has a probable sequence match with another agent of interest, for example, an opportunistic agent, or a subsequent or parallel infection caused by another agent. In another aspect, the epitope 402 selected has a low probable match with the host, for example, to decrease side effects due to the production of self- or auto-antibodies. The term “host,” as used herein, may include but is not limited to an individual, a person, a patient, and/or virtually any organism requiring management of a disease, disorder, and/or condition. For example, the epitope 402 selected may have a 0-70% sequence match at the amino acid level with the host or the agent 400, or a 0-100% sequence match with the agent. Those having skill in the art will recognize that part of that context in relation to the term “host” is that generally what is desired is a practicably close sequence match to the agent (e.g., HIV-1 or influenza virus), so that the one or more immune system components in use can attack it and a practicably distant sequence match to the host (e.g., a patient), in order to decrease or render less aggressive or less likely any attack by the immune system components in use on the host. However, it is also to be understood that in some contexts the agent will in fact constitute a part of the host (e.g., when the agent to be eradicated is actually a malfunctioning part of the host, such as in an auto-immune or neoplastic disease), in which case that part of the host to be eradicated will be treated as the “agent,” and that part of the host to be left relatively undisturbed will be treated as the “host.” In another aspect, the epitope 402 selected has a sequence match with the agent, for example, a high sequence match, or a relatively higher sequence match with other agents compared to the host, or a 0-100% sequence match with the agent 400. The term “sequence match,” as used herein, includes both sequence matching at the nucleic acid level and/or at the protein or polypeptide level. In an embodiment, the epitope 402 selected has a low probable sequence match with the host. In another embodiment, the epitope 402 selected has a high sequence match with other agents.

In molecular biology, the terms “percent sequence identity,” “percent sequence homology” or “percent sequence similarity” are sometimes used interchangeably. In this application the terms are also often used interchangeably, unless context dictates otherwise.

In another aspect, the epitope 402 selected has a likely and/or a probable sequence match with other epitopes, for example, including, but not limited to, the epitope 402 having a structural sequence match, a functional sequence match, a similar functional effect, a similar result in an assay and/or a combination. Structural comparison algorithms and/or 3-dimensional protein structure data may be used to determine whether two proteins or presented fragments thereof may have a structural sequence match. In another example, the epitope 402 may have a functional match and/or share a similar functional effect with epitopes of interest. In this example, the epitope 402 may have a lower probable sequence match but may still exert the same functional effect. In another example, the epitope 402 and/or other epitopes of interest may have a lower probable sequence match but may share similar activities, for example, enzymatic activity and/or receptor binding activity, e.g., as determined by use of an assay.

In another aspect, the epitope 402 selected may be an immunological effective determinant, for example, the epitope 402 may be weakly antigenic, however it may invoke an effective immune response relating to, for example, the nature and/or the type of the immune response component it evokes. In another aspect, the epitope 402 may exert a similar effect on the immune response; for example, the epitope 402 selected may be part of the antigenic structure of an agent unrelated to the disease or disorder in question; however, it may exert a substantially similar effect on the immune system as measured by, for example, the type, the nature, and/or the time-interval of the immune response.

In one aspect, a sequence match with an entity may be determined by, for example, calculating the percent identity and/or percent similarity between epitopes and/or between the epitope 400 and the host. In one aspect, the percent identity between two sequences may be calculated by determining a number of substantially similar positions obtained after aligning the sequences and introducing gaps. For example, in one implementation the percent identity between two sequences is treated as equal to (=) a number of substantially similar positions/total number of positions×100. In this example, the number and length of gaps introduced to obtain optimal alignment of the sequences is considered. In another aspect, the percent identity between two sequences at the nucleic acid level may be determined by using a publicly available software tool such as BLAST, BLAST-2, ALIGN and/or DNASTAR software. Similarly, the percent identity between two sequences at the amino acid level may be calculated by using publicly available software tools such as, for example, Peptidecutter, AACompSim, Find Mod, GlycoMod, InterProtScan, DALI and/or tools listed on the ExPasy Server (Expert Protein Analysis System) Proteomics Server at http://www.expasy.org/. In some embodiments, the percent identity at the nucleic acid level and/or at the amino acid level are determined.

In one aspect, string-matching algorithms may be used to identify homologous segments, for example, using FASTA and BLAST. In another aspect, sequence alignment based on fast Fourier transform (FFT) algorithms may be used to rapidly identify homologous segments. In yet another aspect, iterative searches may be used to identify and select homologous segment. Searches may be used not only to identify and select shared epitopes but also to identify epitopes that have the least homology with human sequences. Additional information may be found in Katoh et al., MAFFT: a novel method for rapid multiple sequence alignment based on fast Fourier transform, Nucleic Acids Research, 30(14):3059-66 (2002) which is incorporated herein by reference.

A number of large-scale screening techniques may be used to identify and select the designed antibody, for example, the antibody designed may be selected by using optical fiber array devices capable of screening binding molecules. Additional information may be found in U.S. Patent Application No. 20040132112 to Kimon et al., which is hereby incorporated by reference.

It will be appreciated by those skilled in the art that the epitope 402 selected need not be limited to a matching sequence displayed by the agent 400. In one aspect, a meta-signature and/or a consensus sequence may be derived based on any number of criteria. In one aspect, the meta-signature may be derived by analysis of data from sources such as, for example, antigenic evolution, genetic evolution, antigenic shift, antigenic drift, data from crystal structure, probable match with a host, probable match with other strains, and/or strength of the immunogenic response desired. The meta-signature may include new sequences and/or may exclude some sequences. For example, it may include silent mutations, mismatches, a spacer to bypass a hotspot or a highly mutagenic site, predicted changes in the sequence, and/or may include epitopes from multiple agents, thus providing protection from multiple agents. As another example, the meta-signature may exclude sequences, such as, for example, including, but not limited to, mutagenic sequences and/or sequences with a high percentage match to the host.

In one aspect, the predicted changes in the epitope 402 may be determined by analysis of past variations observed and/or predicted in the agent 400 (e.g., FIG. 5). Computational analysis can be used to determine regions showing sequence variations and/or hot spots. In one aspect, high speed serial passaging may be performed computationally mimicking the serial passaging that occurs naturally with a production of a new strain of the agent 400. It will be appreciated by those of skill in the art that the hot spots need not be identified by examining the epitope 402, and/or by examining the epitope 402 in context with the agent 400. Information pertaining to hot spots can also be extrapolated by performing sequence analysis of other agents and/or domain analysis of such other agents. For example, in one implementation, the epitope 402 may be part of a domain shared between multiple agents some of which may lack the epitope 402 of interest. Information pertaining to hotspots identified in the domain of the other agents may be of practical use in determining the meta-signature.

In one aspect, one or more sets and/or subsets of epitopes may be formed. The nature and type of criteria used to form the sets and/or subsets will depend, for example, on the nature and type of the agent 400, the duration of the immune response desired (e.g., short-term immunity, or long-term immunity), the nature of the immune response desired (e.g., weak, moderate, or strong), the population to be protected (e.g., presence and/or recency of varying degrees of prior exposure) and the like. The sets and/or subsets so formed may accept input either robotically or from a user (e.g., from a manufacturer of immune response components, from wet lab and/or medical personnel).

The pattern changes predicted in an epitope 402 may be supplemented, for example, by other methodology, statistical analysis, historical data, and/or other extrapolations of the type utilized by those having skill in the art. The knowledge of these predicted pattern changes represents an arsenal in the design and/or selection of the immune response components. The predicted pattern changes may be used to determine the progression of the changes in the immune response component required to manage such changes. Inferring the pattern changes in an epitope 402 and using the information to modulate the progressing response may help manage the response more effectively. For example, the pattern changes may be used to provide a timeline of when the therapy could be changed, what therapy should constitute the change, or the duration of the change. As a more specific example, one reason why Type-1 Human Immunodeficiency Virus (HIV-1) is able to eventually kill its host is that the virus mutates its antigenic signature-profile significantly faster than the human immune system can track and respond to these mutations. In a specific implementation of the subject matter described herein, a sample of HIV-1 is taken from a patient at a point in time and computational biological techniques are used to infer likely mutations of the antigenic signature-profile of the virus at future times. Techniques such as cloning are then utilized to synthesize immune system-activating aspects of the anticipated-future HIV strains, and thereafter replicative techniques are utilized to rapidly generate copious amounts of one or more immune system components (e.g., antibodies) that are keyed to the likely future generation of the patient's particular strain and sub-strain(s) of HIV-1. Once prepared, the immune system components are then administered to the patient and thus are present and waiting for the HIV-1 viral quasispecies when it mutates into the anticipated new forms and/or attempts to proliferate these forms. If the HIV-1 quasispecies mutates as anticipated, the preloaded immune response components successfully negate the mutated quasispecies, thereby likely greatly reducing the patient's viral load—and crucially suppressing the likelihood of further mutation, since the virion population of mutated forms never becomes substantial. In another implementation, the mutational history of the HIV-1 quasispecies is closely tracked, and once the actual mutational direction has been determined, high-speed techniques are utilized to generate immune system components capable of effective suppression of the mutated viral quasispecies, significantly more rapidly than the virus is able to effectively mutate and thus ‘escape’ from the suppressive therapy.

In one aspect, the epitope 402 selected for designating the immune response component may be synthetically made and/or derived from the agent 400. In one embodiment, the epitope 402 selected is derived from an agent 400, extracted from an individual desiring treatment and/or an individual found resistant to that agent. In one aspect, the epitope 402 selected for designating the immune response component may include multiple copies of the exact same epitope and/or multiple copies of different epitopes.

In one aspect, the meta-signature includes sequences matching adjacent and/or contiguous sequences. In another aspect, the meta-signature includes non-adjacent sequences. For example, it will be appreciated by those of skill in the art that peptide splicing and/or proteosomal processing of an epitope that occurs naturally may result in the formation of a new epitope, for example, a non-linear epitope. In this example, proteosomal processing may result in the excision of sequences and the transposing non-contiguous sequences to form the non-linear epitope. Additional information may be found in Hanada et al., Immune recognition of a human renal cancer antigen through post-translational protein splicing, Nature 427:252 (2004), and Vigneron et al., An antigenic peptide produced by peptide splicing in the Proteosome, Science 304:587 (2004) hereby incorporated by reference herein in their entireties.

Additionally, it will also be appreciated by those of skill in the art that the meta-signature may include sequences displayed on two different parts of the agent 400. For example, non-adjacent sequences may appear adjacent each other when the protein is folded. In this aspect, the meta-signature may include non-adjacent sequences for identifying the meta-signature. Furthermore, the meta-signature may include non-adjacent sequences corresponding to a specific conformational state of a protein. Immune response components designed to bind such sequences may be specific to the conformational state of the protein. 3-D and/or crystal structure information may also be used to designate the meta-signature. In one aspect, the meta-signature may include multiple sets of epitopes targeting a predicted pattern change and/or an observed pattern change. For example, multiple sets of epitopes may be designed for vaccination and/or for production of immune response components.

Techniques for epitope mapping are known in the art and herein incorporated by reference. For example, FACS analysis and ELISA may be used to investigate the binding of antibodies to synthetic peptides including at least a portion of the epitope. Epitope-mapping analysis techniques, Scatchard analysis and the like may be used to predict the ability of the antibody 404 to bind to the epitope 402 presented on the agent 400, to determine the binding affinity of the antibody 404 or other immune element to the epitope 402, and/or to discern a desirable configuration for the antibody 404 or other immune element.

Continuing to refer to FIG. 5, in one aspect, for example, the sequences of selected epitopes 506, 512, and 518 may be used to design one or more complementary antibodies or other immune elements 524, 522, and 526, respectively. The sequences of selected epitopes 506, 512, and 518 may be used to form monoclonal antibodies, for example, by cloning or by using human-mouse systems. The sequences of selected epitopes 506, 512, and 518 may be amplified using the polymerase chain reaction (PCR) as described in U.S. Pat. Nos. 4,683,195, 4,683,202, and 4,800,159 to Mullis et al. which are incorporated herein in their entirety. In another aspect, a consensus sequence and/or a meta-signature may be designed and amplified. The relevant sequence(s) may be inserted in an expression vector for producing proteins and the expressed protein(s) subsequently used to produce antibodies specific to the selected epitopes. In one aspect, the selected epitopes may be antigenic but may not be directly immunogenic.

Human antibodies may be made, for example, by using a human-mouse system such as, for example, the Xenomouse technology of Abgenix, Inc., (available from Abgenix, Inc., now a division of Amgen Inc., currently located in Fremont, Calif. 94555) and/or the HuMAb Mouse technology of Medarex, Inc., (available from Medarex Inc. currently having corporate headquarters in Princeton, N.J.). Briefly stated, in these systems the host mouse immunoglobulin genes are inactivated and human immunoglobulin genes are inserted in the host. On stimulation with an antigen, such transgenic mice produce fully human antibodies. Subsequently, human monoclonal antibodies can be isolated according to standard hybridoma technology.

Selection of humanized antibodies with higher binding affinities from promising murine antibodies may be performed, for example, by using computer modeling software developed by Queen et al. The antibodies produced by this method include approximately 90% of the pertinent human sequences. The structure of the specific antibody is predicted based on computer modeling and the retaining of key amino acids predicted to be necessary to retain the shape and, therefore, the binding specificity of the complementarity determining regions (CDRs). Thus, key murine amino acids are substituted into the human antibody framework along with murine CDRs. The software may then be used to test the binding affinity of the redesigned antibody with the antigen. Additional information can be found in U.S. Pat. No. 5,693,762 to Queen et al., which is incorporated herein by reference.

The formation of other antibody fragments, such as, for example, Fv, Fab, F(ab′).sub.2 or Fc may be carried out by, for example, phage antibody generated using the techniques as described in McCafferty et al., Phage antibodies: filamentous phage displaying antibody variable domains, Nature 348:552-554 (1990), and Clackson et al., Making Antibody Fragments Using Phage Display Libraries, Nature 352:624-628 (1991) and U.S. Pat. No. 5,565,332 to Hoogenboom et al., which are incorporated herein by reference. Surface plasmon resonance techniques, for instance, may be used to analyze real-time biospecific interactions. Camelized antibodies, deimmunized antibodies and anti-idiotypic antibodies may be selected by techniques known in the art, which are herein incorporated by reference.

In one aspect, the selection of antibodies for modulating the immune response may be based on their function. For example, activating antibodies, blocking antibodies, neutralizing antibodies, and/or inhibitory antibodies may be used to modulate the immune response. Such antibodies may perform one or more functions under the appropriate conditions. In a more specific example, the antibody 404 may be triggered to undergo a conformational change by providing a cofactor and/or by changing the ambient temperature or other ambient conditions, such as overall osmolality or pH or concentration of a particular compound, atom or ion. The conformation change may result in a new function being performed by the antibody 404.

Techniques for purifying antibodies are known in the art and are incorporated herein by reference. The purified complementary antibodies, such as those shown as 530, 528 or 532, may then be made available for therapeutic and/or prophylacetic treatment.

The term “an effective treatment therapy,” as used herein, includes, but is not limited to, the use of immune response components in combination with other antibodies, antibody fragments, and/or in combination with other treatments, including, but not limited to, drugs, vitamins, hormones, medicinal agents, pharmaceutical compositions and/or other therapeutic and/or prophylacetic combinations. In another aspect, the immune response component may be used in combination, for example, with a modulator of an immune response and/or a modulator of an antibody. In one aspect, cocktails of immune response components may be administered, for example, by injection by a subcutaneous, nasal, intranasal, intramuscular, intravenous, intraarterial, intrathecal, intracapsular, intraorbital, intracardiac, transdermal, intradermal, intraperitoneal, transtracheal, subcuticular, intraarticular, subcapsular, subarachnoidal, intraspinal, epidural, intrasternal, infusion, topical, sublingual, and/or enteric route.

The therapeutic effect of the immune response component may be produced by one or more modes of action. For example, in one aspect, the immune response component may produce a therapeutic effect and/or alleviate the symptoms by targeting specific cells and neutralizing them. In another aspect, the immune response component may bind to and/or block receptors present on an agent 400 and/or may directly and/or indirectly block the binding of molecules, such as, for example, cytokines, and/or growth factors, to an agent 400. In another aspect, the therapeutic effect of the immune response component is produced by functioning as signaling molecules. In this example, the immune response component may induce cross-linking of receptors with subsequent induction of programmed cell death.

The immune response component may be engineered to include, for example, one or more effector molecules, such as, for example, drugs, small molecules, enzymes, toxins, radionuclides, cytokines, and/or DNA molecules. In an example, the immune response component may serve as a vehicle for targeting and binding an agent 400 and/or delivering the one or more effector molecules. In one aspect, the immune response component may be engineered to include one or more effector molecules without the natural effector functions of the immune response component.

In another aspect, one or more immune response components may be coupled to molecules for promoting immune system components to eliminate unwanted cells. This technique has been described for the treatment of tumors, viral-infected cells, fungi, and bacteria using antibodies. Additional information may be found in U.S. Pat. No. 4,676,980 to Segal, which is incorporated herein by reference.

Continuing to refer to the figures, in particular FIGS. 4, 5, and 6, depicted is that a mutation 610 in the selected epitope 506 results in a mutated epitope 629. The term “selected epitope” as typically used herein, often constitutes a type of the more general term of presented epitope, unless context indicates otherwise. The generation of the mutated epitope 629 may reduce the binding of the immune response component, for example, the antibody 624. In one aspect, binding could be enhanced by generating a new antibody 628 corresponding to the mutated epitope 629. The frequency of minor antigenic variations may be predicted by examining known and/or predicted mutational hotspots. For example, additional mutations 611 and/or 613 may be predicted by a computer-based method and corresponding antibodies 628 and/or 626, respectively, may be designed to account for such antigenic variations in the mutated epitopes 629 and/or 630, respectively. In one aspect, an effective treatment therapy, may incorporate this knowledge in providing an effective humoral response towards an agent. For example, a cocktail of immune response components may include the antibodies 624, 628, 626, and/or 612 for binding to the selected epitope 506 and/or its predicted mutated versions. In one aspect, the cocktail of one or more antibodies or other immune response components may be supplemented by additional chemicals, drugs, and/or growth factors. In another aspect, the effective treatment therapy may include varying doses of immune response components, for example, a substantially larger or more prolonged or earlier- or later-administered dosage of 626 relative to 624, 628, and/or 612.

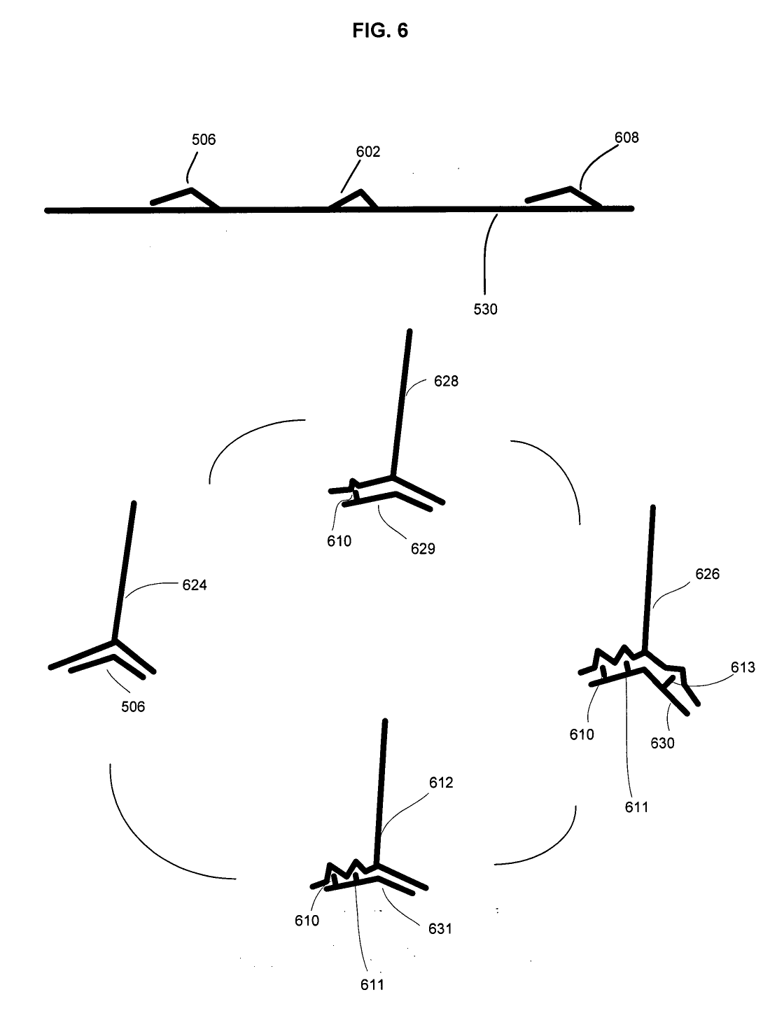

With reference to the figures, and with reference now to FIG. 6, depicted is one aspect of an epitope-antibody interaction showing the occurrence of mutational changes in a selected epitope and corresponding changes in a complementary antibody. For example, the selected epitope 506 may undergo mutational changes. Also by way of example, other epitopes 602 and 608 may not be selected, for example, as the mutation rate for these epitopes may not be appropriate to an embodiment. Mutations may be random and, therefore, non-predictable, or they may be predictable. For example, a mutation may be substantially more predictable based on the occurrence of “hot spots” or known mutational history. The complementary antibody 624 or other immune response component may bind the selected epitope 506, for example, with a usefully-high affinity. However, a sequence change 610 depicted in a mutated selected epitope 629 may reduce the binding affinity of the complementary antibody 624 or other immune response component. A complementary antibody 628 or other immune response component incorporating the mutation alteration may restore the binding affinity, for example, to a usefully-high binding affinity. Similarly, appearance of mutations 610, 611 and 613 may require a new complementary antibody 626 or other immune response component in order to attain a usefully-high binding affinity. Additionally, the appearance of mutations 610 and 611 may require a new complementary antibody 612 or other immune response component. The predictive aspect of the computer system, software and/or circuitry may be used to make mathematically predictable hypotheses regarding the variations and the treatment components required. In one aspect, the complementary antibody or other immune response component need not have a high binding affinity. For example, the complementary antibody 626 or other immune response component may be used to bind and modulate agents with mutations 610, 611 and/or 613.

In another aspect, the antibodies or other immune response components with higher binding affinities may be selected. Numerous techniques exist for enhancing the binding affinity of the antibody or other immune component for the epitope 402. In one aspect, the binding affinity of the antibody or other immune response component for the epitope 402 may be enhanced by constructing phage display libraries from an individual who has been immunized with the epitope 402 either by happenstance or by deliberate immunization. The generation and selection of higher affinity antibodies or other immune response components may also be improved, for example, by mimicking somatic hypermutagenesis, complementarity-determining region (CDR) walking mutagenesis, antibody chain shuffling, and/or technologies such as Xenomax technology (available from Abgenix, Inc., now a division of Amgen, Inc., with headquarters in Fremont, Calif. 94555). In one example, antibodies including introduced mutations may be displayed on the surface of filamentous bacteriophage. Processes mimicking the primary and/or secondary immune response may then be used to select the desired antibodies, for example, antibodies displaying a higher binding affinity for the antigen, and/or by evaluating the kinetics of dissociation. For additional information see, Low et al., Mimicking Somatic Hypermutation: Affinity Maturation Of Antibodies Displayed On Bacteriophage Using A Bacterial Mutator Strain, J. Mol. Biol. 260:359-368 (1996); Hawkins et al. Selection Of Phage Antibodies By Binding Affinity. Mimicking Affinity Maturation, J. Mol. Biol. 226:889-896 (1992), which are incorporated herein by reference.

In another example, the generation and/or selection of higher affinity antibodies may be carried out by CDR walking mutagenesis, which mimics the tertiary immune selection process. For example, saturation mutagenesis of the CDRs of an antibody may be used to generate one or more libraries of antibody fragments which are displayed on the surface of filamentous bacteriophage followed by the subsequent selection of the relevant antibody using immobilized antigen. Sequential and parallel optimization strategies may be used to then select the higher affinity antibody. For additional information see Yang et al., CDR Walking Mutagenesis For The Affinity Maturation Of A Potent Human Anti-HIV-1 Antibody Into The Picomolar Range, J. Mol. Biol 254(3):392-403 (1995), which is incorporated herein by reference in its entirety.

In yet another example, site-directed mutagenesis may be used to generate and select higher affinity antibodies, for example, by parsimonious mutagenesis. In this example, a computer-based method is used to identify and screen amino acid residues included in the one or more CDRs of a variable region of an antibody involved in an antigen-antibody binding. Additionally, in some implementations, the number of codons introduced is such that about 50% of the codons in the degenerate position are wild-type. In another example, antibody chain-shuffling may be used to generate and select higher affinity antibodies. These techniques are known in the art and are herein incorporated by reference.

The dosage of the immune response component may vary and in one aspect may depend, for example, on the duration of the treatment, body mass, severity of the disease, and/or age. Compositions including immune response components may be delivered to an individual for prophylacetic and/or therapeutic treatments. In one aspect, an individual having a disease and/or condition is administered a treatment dose to alleviate and/or at least partially cure the condition expressed by the symptoms. In this example, a therapeutically-effective dose is administered to the patient.

In another aspect, a person's resistance to disease conditions may be enhanced by providing a prophylactically measured dose of at least one antibody. A prophylacetic dose may be provided to, for example, including, but not limited to, a person genetically predisposed to a disease and/or condition, a person traveling to a region where a disease is prevalent, and/or a person wishing to boost that person's immune response.

Optimization of the physico-chemical properties of the immune response component may be improved, for example, by computer-based screening methods. Properties affecting antibody therapeutics may also be improved, such as, for example, stability, antigen binding affinity, and/or solubility. Additional information may be found in U.S. Patent Application No. 20040110226 to Lazar, which is incorporated herein by reference.

With reference to the figures, and with reference now to FIGS. 4, 5, and 6, depicted is one aspect of the antigen-antibody interaction showing the occurrence of mutational changes in the selected epitope 506 and corresponding changes in the complementary antibody 524 or other immune response component. Such mutational changes in the selected epitope 506, for example, may be minor or major in nature. These minor and/or major antigenic variations may render an existing treatment less effective. Thus an effective treatment therapy towards a disease or disorder may include treating the disease or disorder with one or more antibodies designed to anticipate one or more predictable antigenic variations, for example, including, but not limited to, one or more agents or one or more related agents, and/or antibodies directed to epitopes shared with at least two agents. Furthermore, predicting the course of the minor and/or major antigenic variations of an agent and/or the related agents would also be beneficial in designing or selecting these one or more anticipatory antibodies. Additionally, in some implementations, the inclusion of information from SNP databases would be helpful in designing antibodies for binding a selected epitope.

Minor changes in an epitope which do not always lead to the formation of a new subtype may be caused, for example, by point mutations in the selected epitope. In one aspect, the occurrence of point mutations may be localized, for example, to hotspots of the selected epitope. The frequency and/or occurrence of such hotspots may be predicted by one or more computer-based methods. Additionally, methods provide for access to databases including, for example, historical compilations of the antigenic variations of an agent and/or of a selected epitope, for example, from previous endemics and/or pandemics or the natural evolutionary history of the disease. Such information may be part of an epitope profile for charting the progression of the immune response. For example, including, but not limited to, a point mutation in the glutamic acid at position 92 of the NS1 protein of the influenza virus that has been shown to dramatically downregulate activation of cytokines. Such information may be useful in designating a meta-signature.

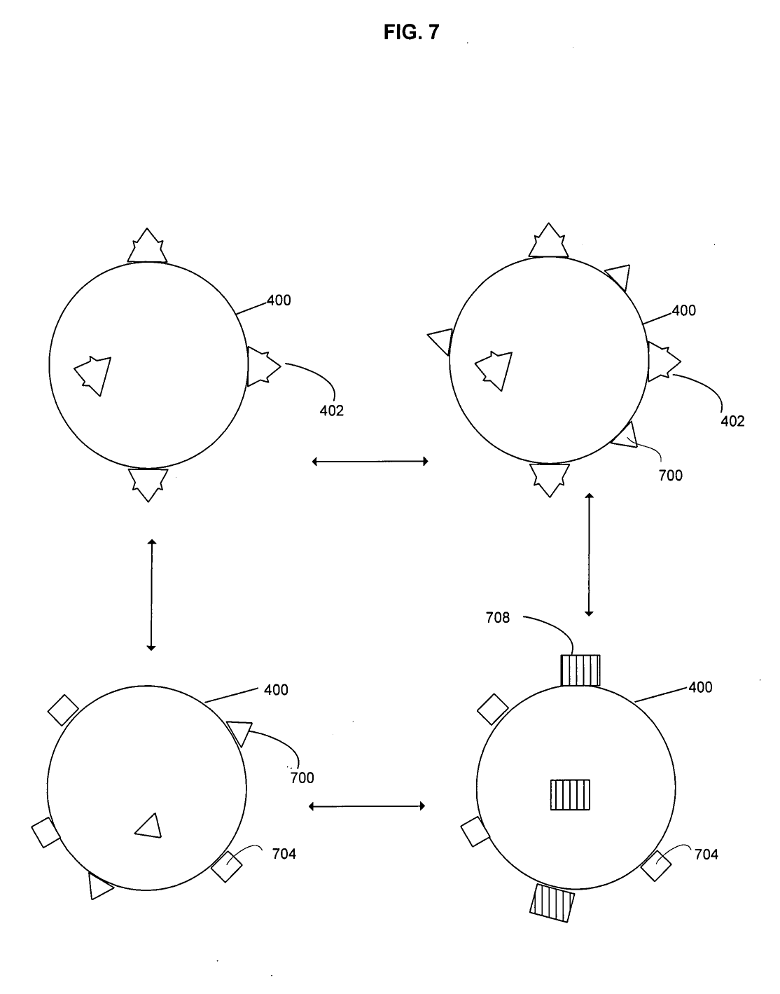

Referring now to FIG. 7, illustrated is one aspect of mutational changes in an epitope displayed by an agent and the corresponding changes in an immune response component, for example, one or more new epitopes 700 and/or 704 may appear on the surface of an agent 400. In one aspect, major changes may occur in the antigenic variants present on the surface of an agent 400 resulting in the formation of a new subtype or sub-strain. The appearance of new epitopes observed, for example, may occur as a result of antigenic shifts, reassortment, reshuffling, rearrangement of segments, and/or swapping of segments and generally marks the appearance of a new virulent and/or pathogenic (sub-)strain of an agent. In one instance, the prediction of the new epitopes may mark the emergence of a new (sub-)strain, a new subtype, and/or the reemergence of an older (sub-)strain. In this instance, natural and/or artificial immune protection in an individual alone may not provide adequate protection. Immune protection and/or humoral protection may be supplemented with, for example, drugs, chemicals or small molecules capable of enhancing, supplanting or favorably interacting with the effects of the pertinent immune response components.

Generally, when major epitopic changes do occur, a larger section of the impacted population succumbs to the infection, sometimes leading to a pandemic. This problem may be alleviated in part, for example, by predicting the appearance of new (sub-)strains and/or subtypes as a result of the appearance of new epitopes and/or the disappearance of existing epitopes. In one aspect, for example, including, but not limited to, the prediction of the new epitopes, attention may be directed towards a subset of genes, for example, important for the overall Darwinian fitness and/or replication and/or infectivity of an agent. For example, examining the appearance of new subtypes of influenza virus type A shows that the antigenic variations occur for the most part as a result of mutations in the neuraminidase and/or hemagglutinin genes.

In another aspect, the selected epitope may steer clear of highly variable regions and focus instead on areas having lower probability of mutations. Thus epitopes selected may circumvent hotspots of antigenic variations and target other specific regions of an agent, such as, for example, the receptor-binding site on the surface of the agent. In another example, the selected epitope may not be readily accessible to the immune response component, for example, the receptor-binding site may be buried deep in a ‘pocket’ of a large protein and may be surrounded by readily accessible sequences exhibiting higher level(s) of antigenic variations. In this example, one possibility may include providing small antibody fragments that penetrate the receptor-binding site preventing the agent from binding. In another example, a drug and/or chemical may be used to modify and/or enhance the accessibility of the receptor-binding site. In yet another example, a chemical with a tag may be used to bind to the receptor and the tag then used for binding the immune response component.

In another aspect, the immune response component may be designed so as to circumvent the shape changes in the epitope 402 and provide sufficiently effective binding to the epitope 402 even following mutational change therein. In this example, the antibody or other immune response component designed may include accommodations in its design arising from the prediction of hotspots and/or the mutational changes in the epitope 402.

In one aspect, the size of the immune response component may be manipulated. An immune response component, for example the antibody 404, may be designed to include a practicably minimal binding site required to bind the epitope 402. In another example, the immune response component may be designed for binding to the smallest effective determinant.

In one aspect, an effective treatment therapy towards a disease and/or disorder may include one or more immune response components designed to anticipate and/or treat an antigenic drift and/or an antigenic shift predicted for multiple agents. The agents need not be related to each other, for example, the therapy might be designed for an individual suffering from multiple diseases.

B. Operation(s) and/or Process(es)

Following are a series of flowcharts depicting implementations of processes. For ease of understanding, the flowcharts are organized such that the initial flowcharts present implementations via an overall “big picture” or top-level viewpoint, and thereafter the following flowcharts present alternate implementations and/or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to and/or further details in subsequent flowcharts) generally allows for a more rapid and reliable understanding of the various process implementations.

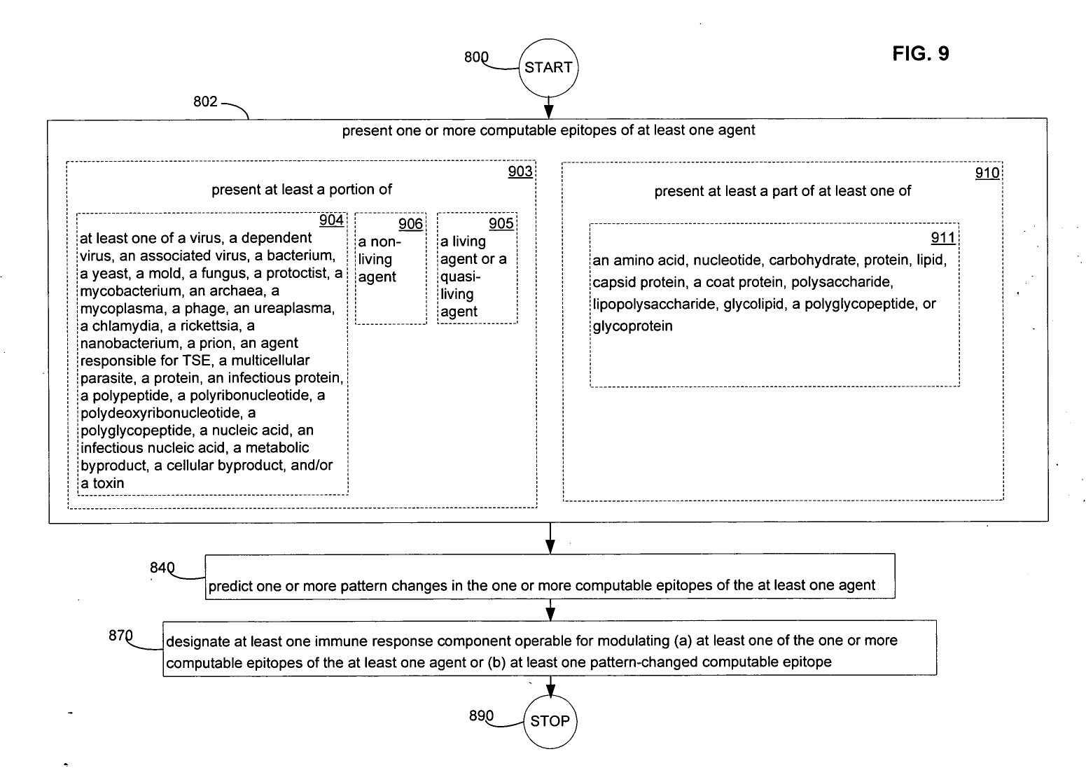

Several of the alternate process implementations are set forth herein by context. For example, as set forth herein in relation to FIG. 9, what is described as method step 904 is illustrated as a list of exemplary qualifications of an agent. Those skilled in the art will appreciate that when what is described as method step 904 is read in the context of what are described as method step 903 and method step 802, it is apparent that the list of exemplary qualifications of the agent, in context, is actually illustrative of an alternate implementation of method step 802 of presenting at least a portion of at least one of a virus, a dependent virus, an associated virus, a bacterium, a yeast, a mold, a fungus, a protoctist, a mycobacterium, an archaea, a mycoplasma, a phage, a ureaplasma, a chlamydia, a rickettsia, a nanobacterium, a prion, an agent responsible for TSE, a multicellular parasite, a protein, an infectious protein, a polypeptide, a polyribonucleotide, a polydeoxyribonucleotide, a polyglycopeptide, a nucleic acid, an infectious nucleic acid, a metabolic byproduct, a cellular byproduct, and/or a toxin. Likewise, when what is described as method step 905 is read in the context of what are described as method step 903 and method step 802, it is apparent that, in context, method step 905 is actually illustrative of an alternate implementation of method step 802 of presenting at least a portion of a living agent and/or a quasi-living agent. Likewise again, when what is described as method step 906 is read in the context of what are described as method step 903 and method step 802, it is apparent that, in context, method step 906 is actually illustrative of an alternate implementation of method step 802 of presenting at least a portion of a non-living agent. Contextual readings such as those just set forth in relation to method steps 904, 905, and 906 are within the ambit of one having skill in the art in light of the teaching herein, and hence are not set forth verbatim elsewhere herein for sake of clarity and/or brevity.

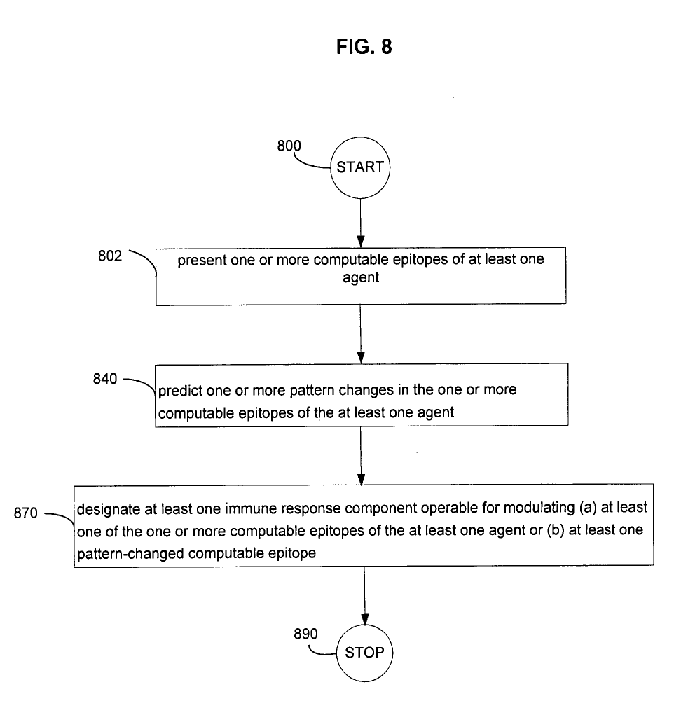

With reference now to FIG. 8, depicted is a high-level logic flowchart of a process. Method step 800 shows the start of the process. Method step 802 depicts presenting one or more computable epitopes of at least one agent. Method step 840 depicts predicting one or more pattern changes in the one or more computable epitopes of the at least one agent. For example, previous pattern changes known and/or predicted may be used to extrapolate future progressions of the pattern changes that may be observed in the one or more determinants of the agent. Method step 870 depicts designating at least one immune response component operable for modulating (a) at least one of the one or more computable epitopes of the at least one agent and/or (b) at least one pattern-changed computable epitope. The immune response components so designated may include those for managing a disease, a condition for managing a response, for example, a biological response. Method step 890 shows the culmination or end of the process.

With reference now to FIG. 9, depicted is a high-level logic flowchart depicting alternate implementations of the high-level logic flowchart of FIG. 8. Illustrated is that in various alternate implementations, method step 802 may include at least one of sub-steps 903 and/or 910. Method step 903 depicts presenting at least a portion of the agent. Method step 910 depicts presenting at least a part of at least one computable epitope. Method step 903 depicts some exemplary qualifications of an agent and may include at least one of sub-steps 904, 905, and/or 906. Method step 910 depicts some exemplary qualifications of a computable epitope and may include sub-step 911. As depicted, method step 904 may include presenting at least a portion of at least one of a virus, a dependent virus, an associated virus, a bacterium, a yeast, a mold, a fungus, a protoctist, a mycobacterium, an archaea, a mycoplasma, a phage, a ureaplasma, a chlamydia, a rickettsia, a nanobacterium, a prion, an agent responsible for TSE, a multicellular parasite, a protein, an infectious protein, a polypeptide, a polyribonucleotide, a polydeoxyribonucleotide, a polyglycopeptide, a nucleic acid, an infectious nucleic acid, a metabolic byproduct, a cellular byproduct, and/or a toxin. The agent may include a living agent and/or a quasi-living agent as depicted in method step 905 and/or a non-living agent as depicted in method step 906. Method step 911 depicts presenting at least a part of at least one of an amino acid, a nucleotide, a carbohydrate, a protein, a lipid, a capsid protein, a coat protein, a polysaccharide, a lipopolysaccharide, a glycolipid, a polyglycopeptide, and/or a glycoprotein. It will also be appreciated by those skilled in the art that method step 802 may include accepting input related to, for example, the agent, the one or more computable epitopes and/or other relevant criteria, such as, but not limited to, a size or configuration of the computable epitope, a type of the computable epitope, a nature of the disease, a disorder and/or a condition requiring management, and/or a sensitivity of a group requiring management.

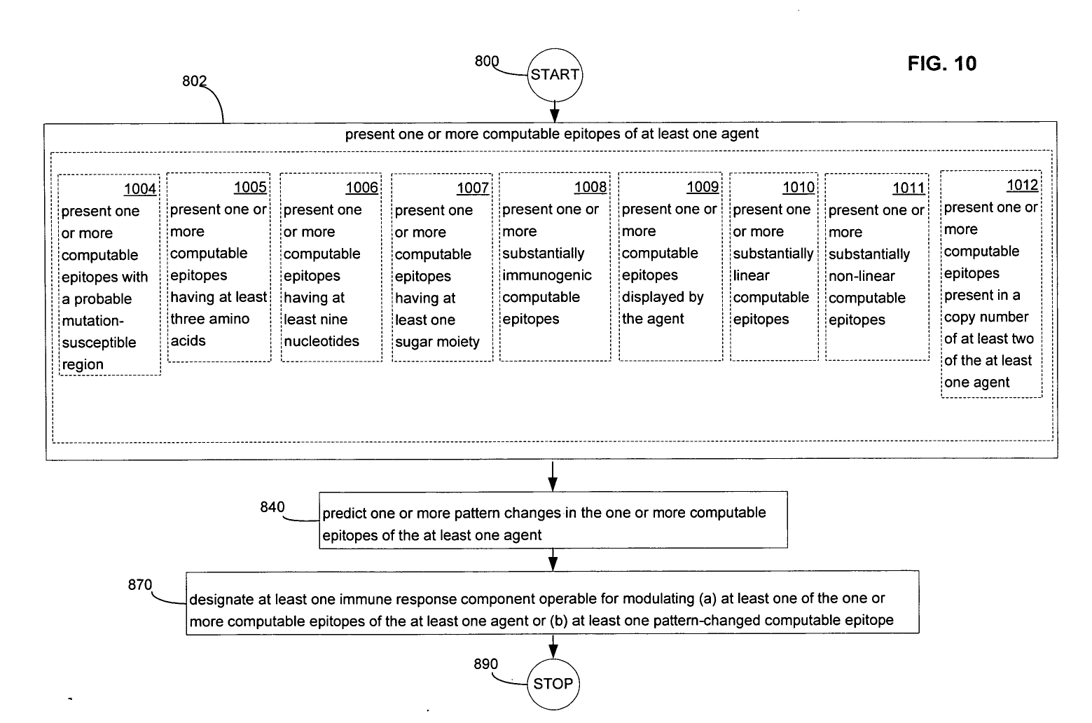

With reference now to FIG. 10, depicted is a high-level logic flowchart depicting alternate implementations of the high-level logic flowchart of FIG. 8. In various alternate implementations, method step 802 may include at least one of method steps 1004, 1005, 1006, 1007, 1008, 1009, 1010, 1011, and/or 1012. Method step 1004 depicts presenting one or more computable epitopes with a probable mutation-susceptible region (e.g., a mutagenic ‘hot spot’ or a highly mutable region). Method step 1005 depicts presenting one or more computable epitopes having at least three amino acids. Method step 1006 depicts presenting one or more computable epitopes having at least nine nucleotides. It will be appreciated by those of skill in the art that the term “amino acid” may include but is not limited to complete and/or partial amino acids, amino acid residues, amino acid moieties, and/or components thereof. It will be appreciated by those of skill in the art that the term “nucleotide” may include but is not limited to complete and/or partial nucleotides, nucleotide residues, nucleotide moieties, and/or components thereof. Method step 1007 depicts presenting one or more computable epitopes having at least one sugar moiety. Method step 1008 depicts presenting one or more substantially immunogenic computable epitopes (e.g., a computable epitope distinguished by the occurrence of an immune response directed towards it). Method step 1009 depicts presenting one or more computable epitopes displayed by the agent. Method step 1010 depicts presenting one or more substantially linear computable epitopes. Method step 1011 depicts presenting one or more substantially non-linear computable epitopes displayed by the agent (e.g., on a surface of the agent, on a peculiarity of its surface, adjacent to a hotspot, and/or adjacent to a cleavage site). Method step 1012 depicts presenting one or computable epitopes present in a copy number of at least two of the at least one agent.

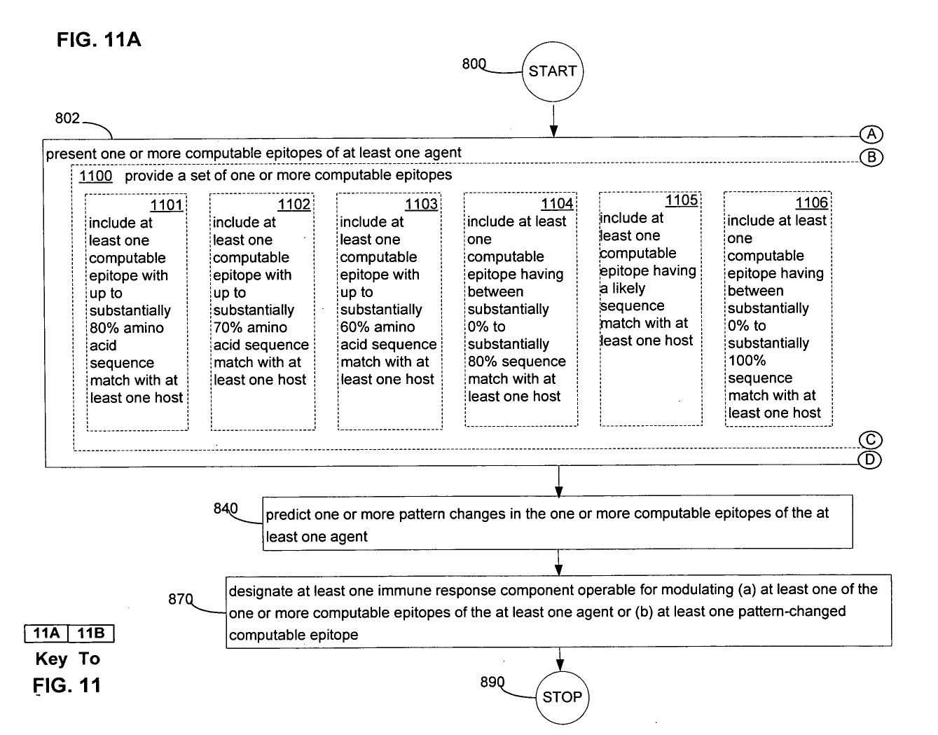

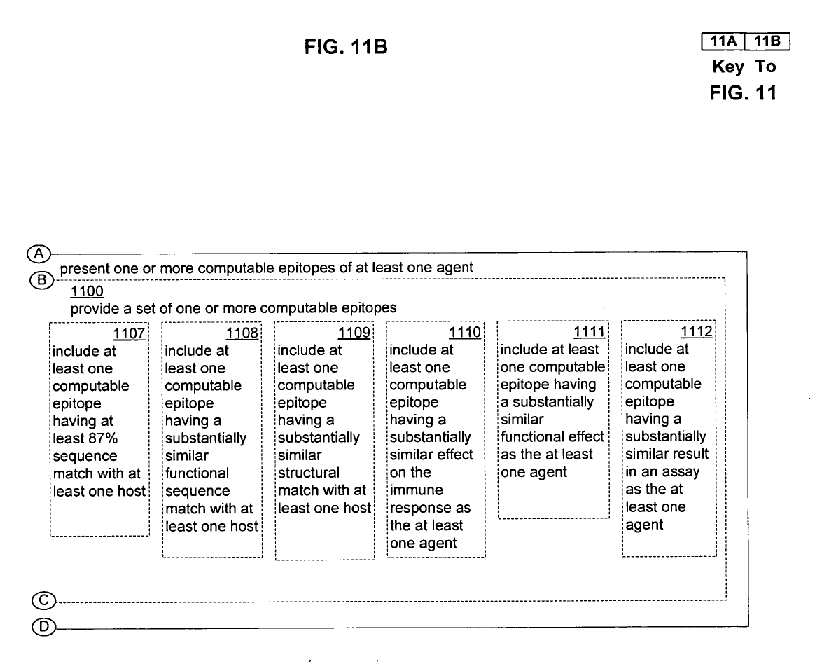

With reference to the figures, and with reference now to FIG. 11, depicted is a high-level logic flowchart exhibiting alternate implementations of the high-level logic flowchart of FIG. 8. Shown is that in alternate implementations, method step 802 may include sub-step 1100. The presentation of one or more computable epitopes may include providing a set of one or more computable epitopes method step 1100 (e.g., a group of one or more computable epitopes). Depicted here is that in various alternate implementations method step 1100 may include at least one of sub-steps 1101, 1102, 1103, 1104, 1115, 1106, 1107, 1108, 1109, 1110, 1111, and/or 1112 which depict various criteria for forming a set. Method step 1101 depicts providing a set including at least one computable epitope with up to substantially 80% amino acid sequence match with the at least one agent and/or a host. Method step 1102 depicts providing a set including at least one computable epitope with up to substantially 70% amino acid sequence match with the at least one agent and/or a host. Method step 1103 depicts providing a set including at least one computable epitope with up to substantially 60% amino acid sequence match with the at least one agent and/or a host. Method step 1104 depicts providing a set including at least one computable epitope having between substantially 0% to substantially 80% sequence match (e.g., amino acid and/or nucleotide sequence match) with the at least one agent and/or a host (e.g., a 0% practicable sequence match is sometimes useful, for example, in implementations including, but not limited to, when the sequence desired is one that elicits a practicably relatively lower auto-immune response in the host and/or when the sequence desired is one that has a relatively lower crossover with sequences of another agent). Method step 1105 depicts providing a set including at least one computable epitope having a likely sequence match with the at least one agent and/or a host (e.g., a probable sequence match). Method step 1106 depicts providing a set including at least one computable epitope having between substantially 0% to substantially 100% sequence match with the at least one agent and/or a host (e.g., a 0% practicable sequence match is sometimes useful, for example, in implementations including, but not limited to, when the sequence desired is one that elicits a practicably lower auto-immune response in the host and/or when the sequence desired is one that has a practicably relatively lower crossover sequence match with another agent; a 100% practicable sequence match is sometimes useful, for example, in implementations including, but not limited to, when the sequence desired is one that elicits a practicably higher immune response in the host against the agent, and/or when the sequence desired is one that has a practicably relatively higher crossover sequence match with the host (e.g., an irretrievably infected host), for example, when eradication of the host needs to be accomplished in an environmentally-friendly manner). Method step 1107 depicts providing a set including at least one computable epitope having at least 87% sequence match with the at least one agent and/or a host (e.g., amino acid and/or nucleotide sequence match). Method step 1108 depicts providing a set including at least one computable epitope having a substantially similar functional sequence match with the at least one agent and/or a host (e.g., a function such as enzymatic activity, binding, blocking, and/or activating other proteins). Method step 1109 depicts providing a set including at least one computable epitope having a substantially similar structural match with the at least one agent and/or a host. Method step 1110 depicts providing a set including at least one computable epitope having a substantially similar effect on the immune response as the at least one agent. Method step 1111 depicts providing a set including at least one computable epitope having a substantially similar functional effect as the at least one agent. Method step 1112 depicts providing a set including at least one computable epitope having a substantially similar result in an assay as the at least one agent. It will also be appreciated by those skilled in the art that method step 1100 may include one or more sub-steps wherein the set is provided by other relevant criteria (e.g., biological criteria, geographical criteria or other substantive criteria). It will also be appreciated by those skilled in the art that method step 1100 may include accepting input for the selection of the sub-steps.

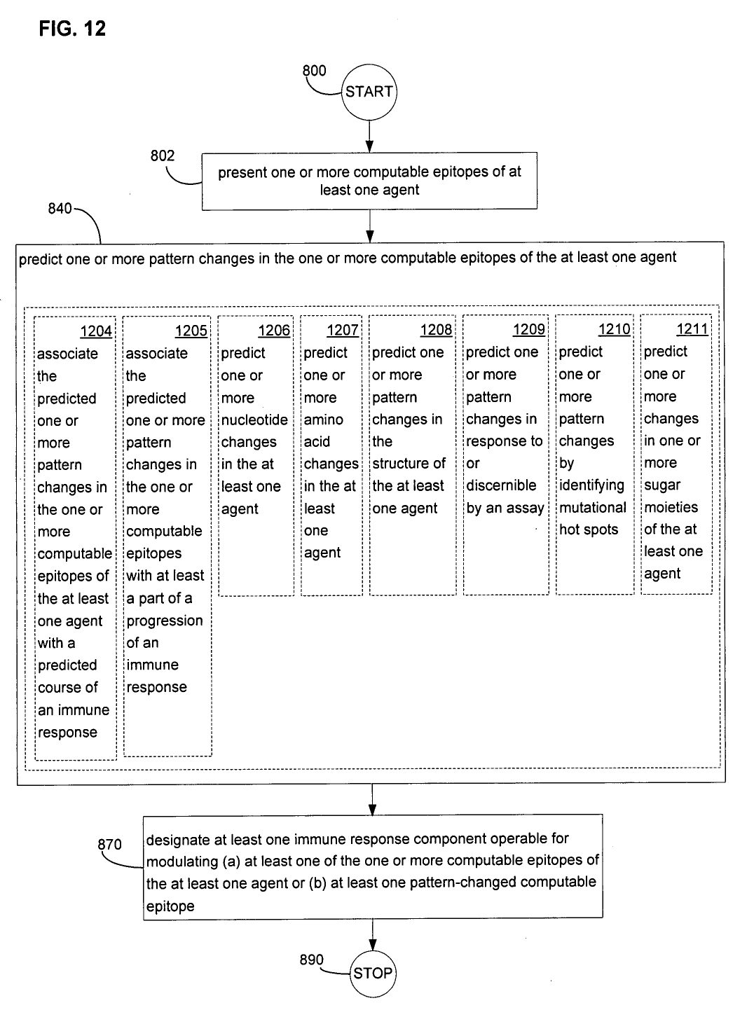

With reference to the figures, and with reference now to FIG. 12, depicted is a high-level logic flowchart depicting alternate implementations of the high-level logic flowchart of FIG. 8. Shown is that in various alternate implementations method step 840 may include at least one of sub-steps 1204, 1205, 1206, 1207, 1208, 1209, 1210 and/or 1211. Method step 1204 shows associating the predicted one or more pattern changes in the one or more computable epitopes of the at least one agent with a predicted course of an immune response. Method step 1205 shows associating the predicted one or more pattern changes in the one or more computable epitopes with at least a part of a progression of an immune response (e.g., an immune response that is treatable, and/or an immune response that is intense). Method step 1206 shows predicting one or more nucleotide changes in the at least one agent (e.g., a nucleotide change associated with a conformational change, a functional change and/or associated with latency). Method step 1207 shows predicting one or more amino acid changes in the at least one agent (e.g., an amino change associated with an enzymatic activity, binding and/or other functions). Method step 1208 shows predicting one or more pattern changes in the structure of the at least one agent (e.g., changes in the glycosylated protein, and/or domain swapping). Method step 1209 shows predicting one or more pattern changes in response to or discernible by an assay (e.g., binding, inhibition, and/or activation assays). Method step 1210 shows predicting one or more pattern changes by identifying mutational hot spots. Method step 1211 depicts predicting one or more changes in one or more sugar moieties of the at least one agent.

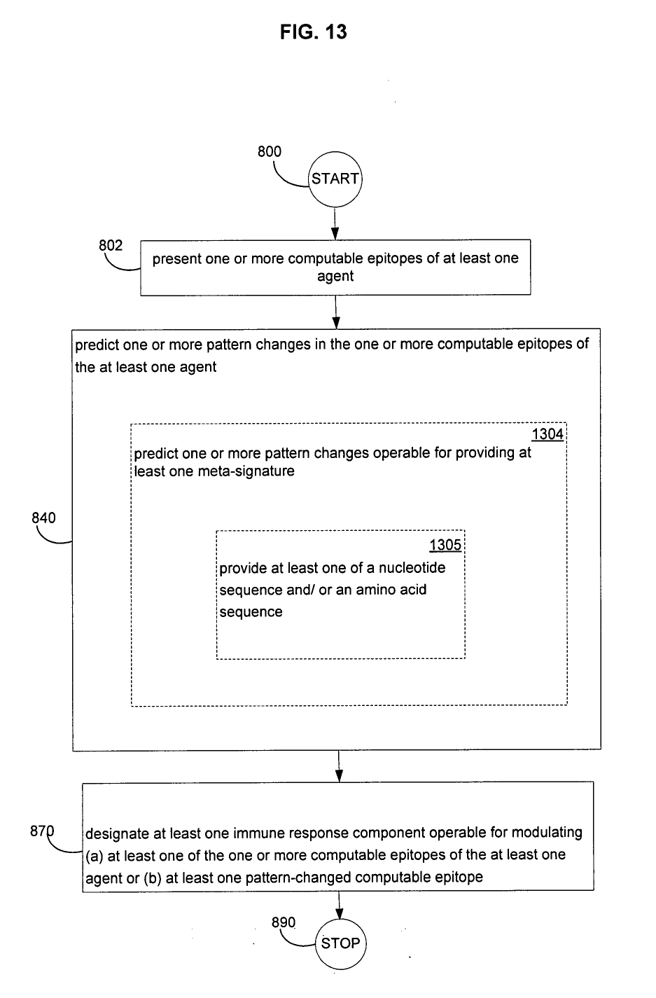

With reference to the figures, and with reference now to FIG. 13, depicted is a high-level logic flowchart depicting alternate implementations of the high-level logic flowchart of FIG. 8. Shown is that in various alternate implementations method step 840 may include sub-step 1304. Method step 1304 depicts predicting one or more pattern changes operable for providing at least one meta-signature (e.g., at least one sequence shared by one or more agents for modulating an immune response, and/or at least one consensus sequence derived from one or more agents for modulating an immune response). In one alternate implementation, method step 1304 may include method step 1305 which depicts providing at least one meta-signature by providing at least one of a nucleotide sequence and/or an amino acid sequence.

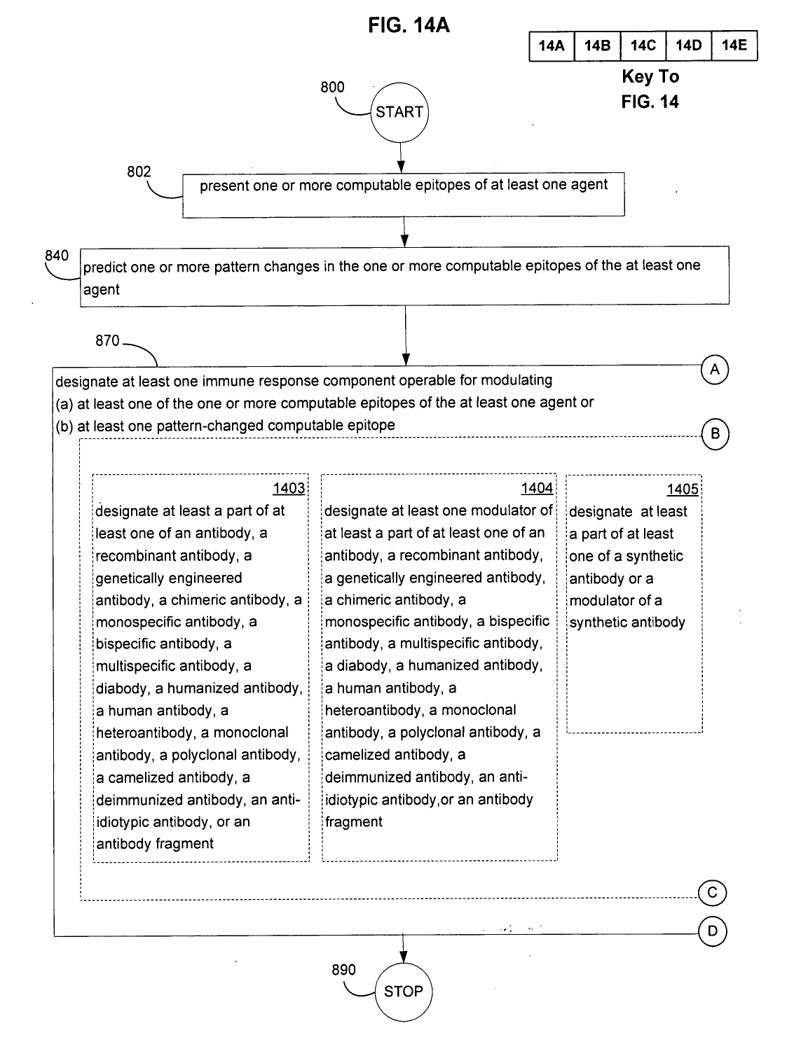

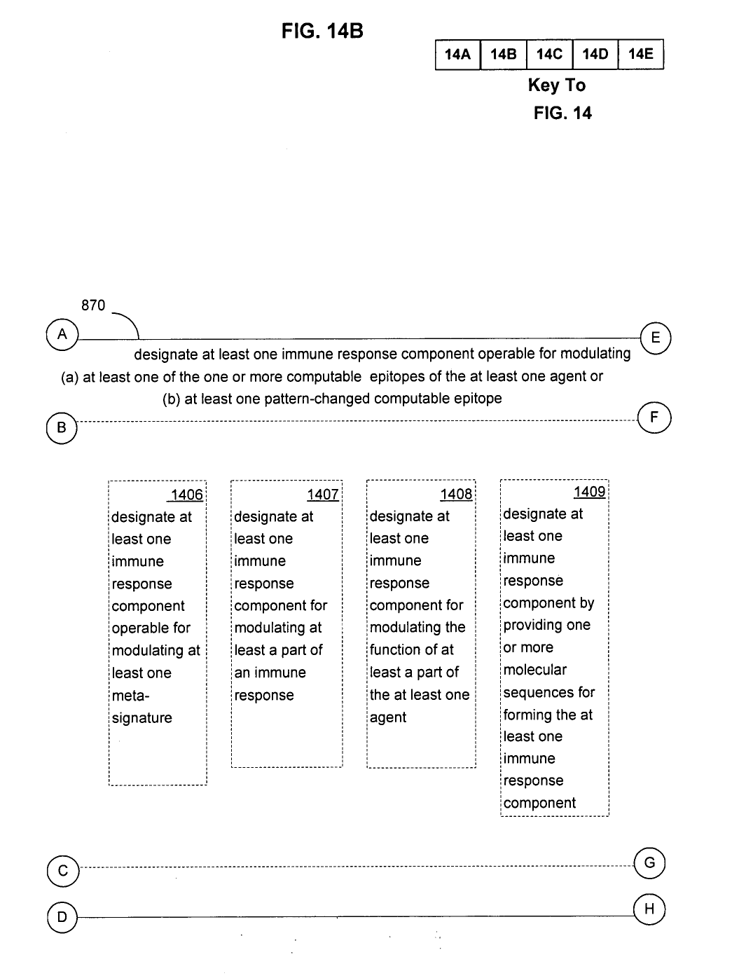

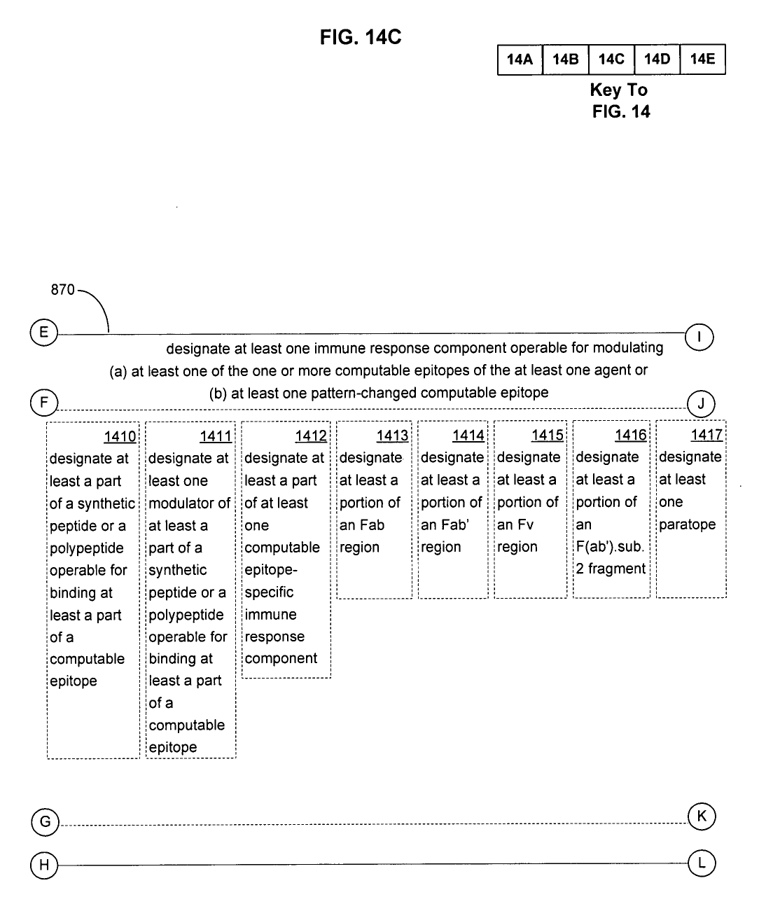

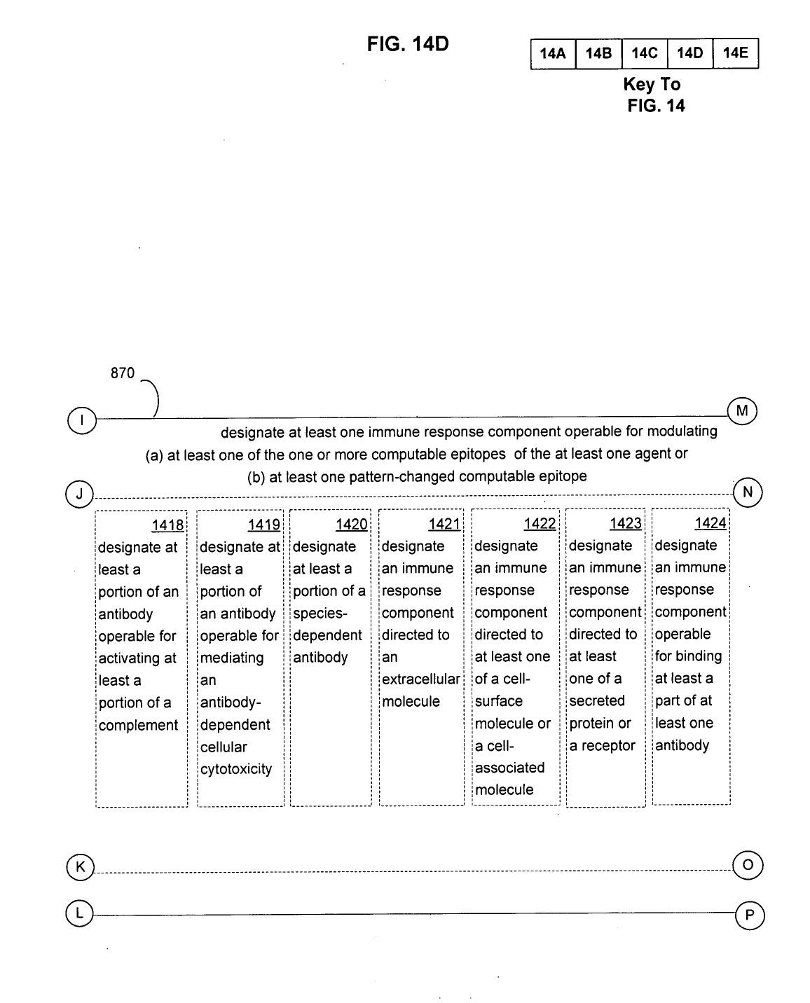

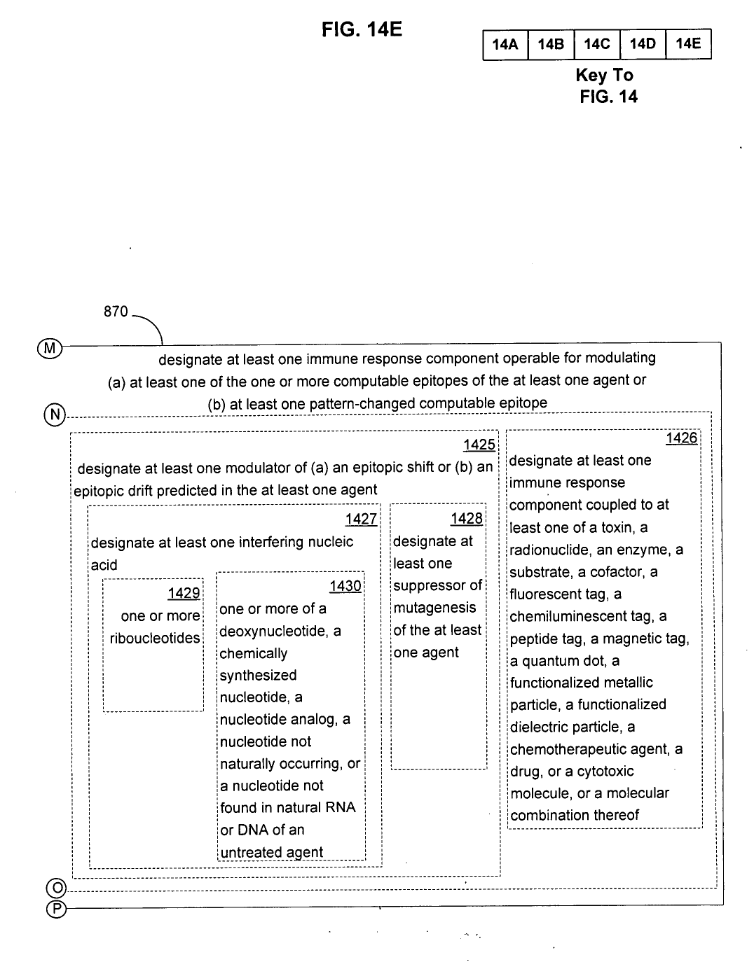

With reference to the figures, and with reference now to FIG. 14, depicted is a high-level logic flowchart depicting alternate implementations of the high-level logic flowchart of FIG. 8. Depicted is that in various alternate implementations method step 870 may include at least one of method steps 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, and/or 1426. Method step 1403 depicts designating at least a part of at least one of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. Method step 1404 depicts designating at least one modulator of at least a part of at least one of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment (e.g., a small molecule, a drug, and/or a compound). Method step 1405 depicts designating at least a part of at least one of a synthetic antibody and/or a modulator of a synthetic antibody. Method step 1406 depicts designating at least one immune response component operable for modulating at least one meta-signature. Method step 1407 depicts designating at least one immune response component for modulating at least a part of an immune response (e.g., an immune response requiring immediate management, and/or an immune response requiring management in the future). Method step 1408 depicts designating at least one immune response component for modulating the function of at least a part of the at least one agent (e.g., blocking and/or inhibiting the function). Method step 1409 depicts designating at least one immune response component by providing one or more molecular sequences for forming the at least one immune response component. Method step 1410 depicts designating at least a part of a synthetic peptide and/or a polypeptide operable for binding at least a part of a computable epitope (e.g., a peptide and/or a polypeptide including modifications, such as, and not limited to, a glycosylated peptide and/or a glycosylated polypeptide). Method step 1411 depicts designating at least one modulator of at least a part of a synthetic peptide and/or a polypeptide operable for binding at least a part of a computable epitope. Method step 1412 depicts designating at least a part of at least one computable epitope-specific immune response component. Method step 1413 depicts designating at least a portion of a Fab region. Method step 1414 depicts designating at least a portion of a Fab′ region. Method step 1415 depicts designating at least a portion of a Fv region. Method step 1416 depicts designating at least a portion of a F(ab′).sub.2 fragment. Method step 1417 depicts designating at least one paratope. Method step 1418 depicts designating at least a portion of an antibody operable for activating at least a portion of a complement. Method step 1419 depicts designating at least a portion of an antibody operable for mediating an antibody-dependent cellular cytotoxicity. Method step 1420 depicts designating at least a portion of a species-dependent antibody. Method step 1421 depicts designating an immune response component directed to an extracellular molecule. Method step 1422 depicts designating an immune response component directed to at least one of a cell-surface molecule and/or a cell-associated molecule. Method step 1423 depicts designating an immune response component directed to at least one of a secreted protein and/or a receptor. Method step 1424 depicts designating an immune response component operable for binding at least a part of at least one antibody (e.g., when the immune response requiring management is an auto-immune response). Method step 1425 depicts designating at least one modulator of (a) an epitopic shift and/or (b) an epitopic drift predicted in the at least one agent (e.g., a compositional and/or structural shift and/or drift). In one alternate implementation method step 1425 may include at least one of sub-steps 1427 and/or 1428. Method step 1427 depicts designating at least one interfering nucleic acid (e.g., for down-regulating gene activity). In one alternate implementation method step 1427 may include at least one of sub-steps 1429 and/or 1430. Method step 1429 shows that the interfering nucleic acid may include one or more ribonucleotides and method step 1430 depicts that the interfering nucleic acid may include one or more of a deoxynucleotide, a chemically synthesized nucleotide, a nucleotide analog, a nucleotide not naturally occurring, or a nucleotide not found in natural RNA or DNA of an untreated agent. Method step 1428 depicts designating at least one suppressor of mutagenesis of the at least one agent (e.g., a chemical, a compound, and/or a drug that decreases the mutation rate). Method step 1426 depicts designating at least one immune response component coupled to at least one of a toxin, a radionuclide, an enzyme, a substrate, a cofactor, a fluorescent tag, a chemiluminescent tag, a peptide tag, a magnetic tag, a quantum dot, a functionalized metallic particle, a functionalized dielectric particle, a chemotherapeutic agent, a drug, a cytotoxic molecule, and/or a molecular combination thereof (e.g., the immune response component may be coupled directly to the tag or indirectly coupled to the tag via an entity and/or a moiety).

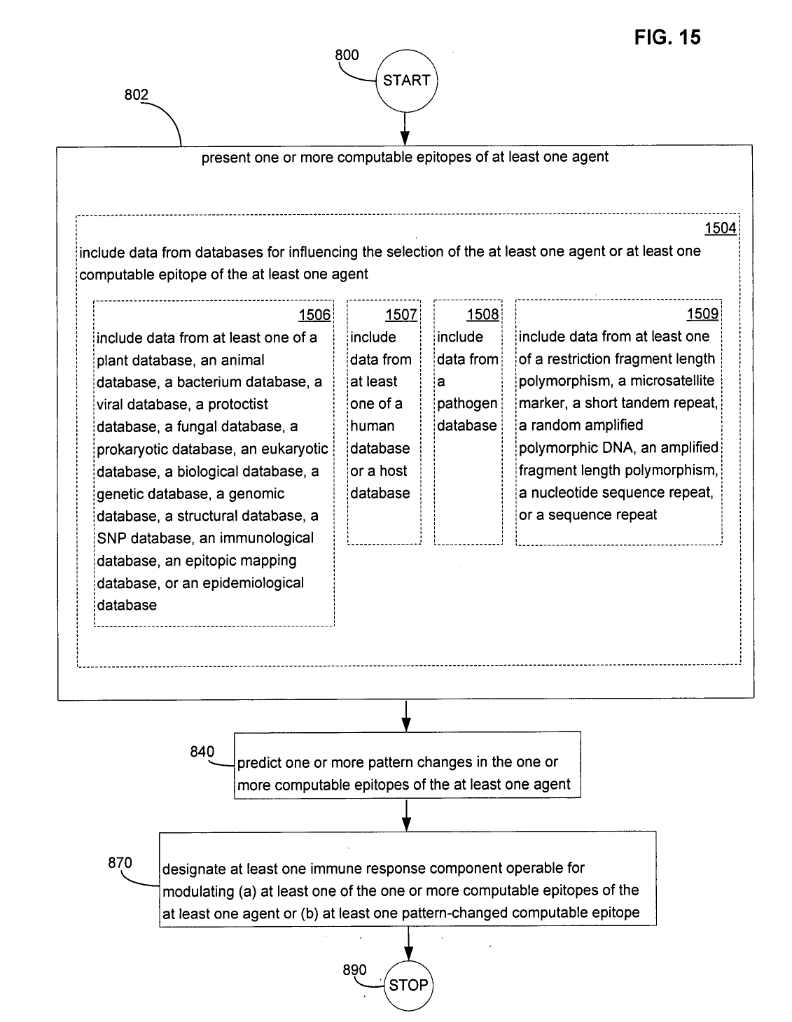

With reference to the figures, and with reference now to FIG. 15, depicted is a high-level logic flowchart depicting alternate implementations of the high-level logic flowchart of FIG. 8. Depicted is that in various alternate implementations method step 802 may include method step 1504. Method step 1504 depicts including data from databases for influencing the selection of the at least one agent or at least one computable epitope of the at least one agent. In various alternate implementations, method step 1504 may include at least one of substeps 1506, 1507, 1508 and/or 1509. Method step 1506 depicts including data from at least one of a plant database, an animal database, a bacterium database, a viral database, a protoctist database, a fungal database, a prokaryotic database, an eukaryotic database, a biological database, a genetic database, a genomic database, a structural database, a SNP database, an immunological database, an epitopic mapping database, and/or an epidemiological database. Method step 1507 depicts including data from at least one of a human database and/or a host database. Method step 1508 depicts including data from a pathogen database. Method step 1509 depicts including data from at least one of a restriction fragment length polymorphism, a microsatellite marker, a short tandem repeat, a random amplified polymorphic DNA, an amplified fragment length polymorphism, a nucleotide sequence repeat, and/or a sequence repeat.

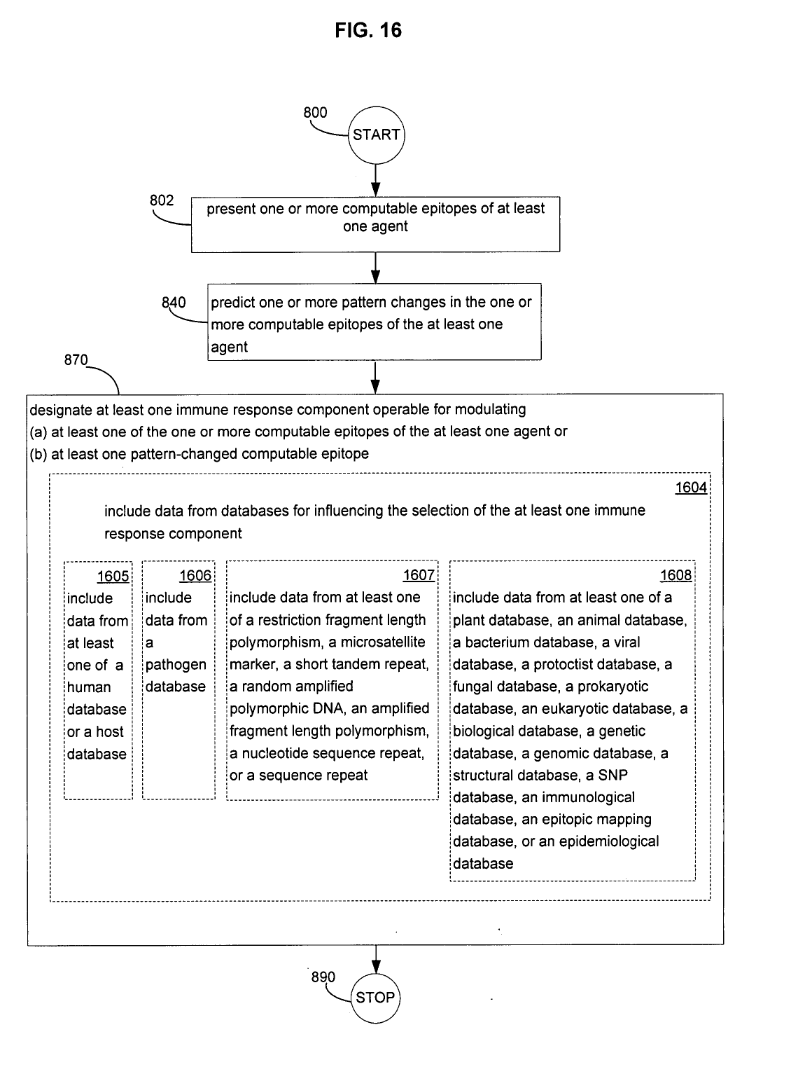

With reference to the figures, and with reference now to FIG. 16, depicted is a high-level logic flowchart depicting alternate implementations of the high-level logic flowchart of FIG. 8. Illustrated is that in various alternate implementations method step 870 may include method step 1604. Method step 1604 depicts including data from databases for influencing the selection of the at least one immune response component. In an alternate implementation method step 1604 may include at least one of sub-steps 1605, 1606, 1607 and/or 1608. Method step 1605 depicts including data from at least one of a human database or a host database. Method step 1606 depicts including data from a pathogen database. Method step 1607 depicts including data from at least one of a restriction fragment length polymorphism, a microsatellite marker, a short tandem repeat, a random amplified polymorphic DNA, an amplified fragment length polymorphism, a nucleotide sequence repeat, and/or a sequence repeat. Method step 1608 depicts including data from at least one of a plant database, an animal database, a bacterium database, a viral database, a fungal database, a protoctist database, a prokaryotic database, an eukaryotic database, a biological database, a genetic database, a genomic database, a structural database, a SNP database, an immunological database, an epitopic mapping database, and/or an epidemiological database.

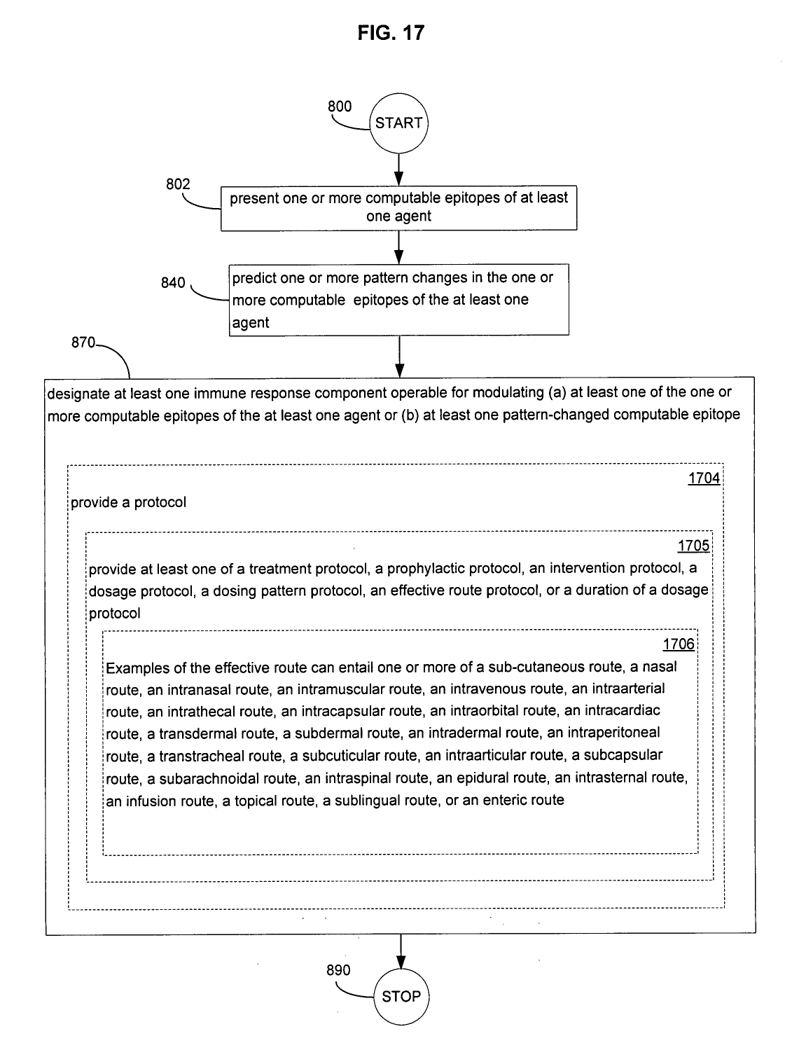

With reference to the figures, and with reference now to FIG. 17, depicted is a high-level logic flowchart depicting alternate implementations of the high-level logic flowchart of FIG. 8. Illustrated is that in various alternate implementations method step 870 may include method step 1704. Method step 1704 depicts providing a protocol (e.g., a scheme, a list of options, and/or a course of action). In one alternate implementation method step 1704 may include substep 1705. Method step 1705 depicts providing at least one of a treatment protocol, a prophylacetic protocol, an intervention protocol, a dosage protocol, a dosing pattern protocol, an effective route protocol, and/or a duration of a dosage protocol. In one alternate implementation method step 1705 may include example-block 1706. Example-block 1706 depicts that examples of the effective route may include one or more of a subcutaneous route, a nasal route, an intranasal route, an intramuscular route, an intravenous route, an intraarterial route, an intrathecal route, an intracapsular route, an intraorbital route, an intracardiac route, a transdermal route, a subdermal route, an intradermal route, an intraperitoneal route, a transtracheal route, a subcuticular route, an intraarticular route, a subcapsular route, a subarachnoidal route, an intraspinal route, an epidural route, an intrasternal route, an infusion route, a topical route, a sublingual route and/or an enteric route.

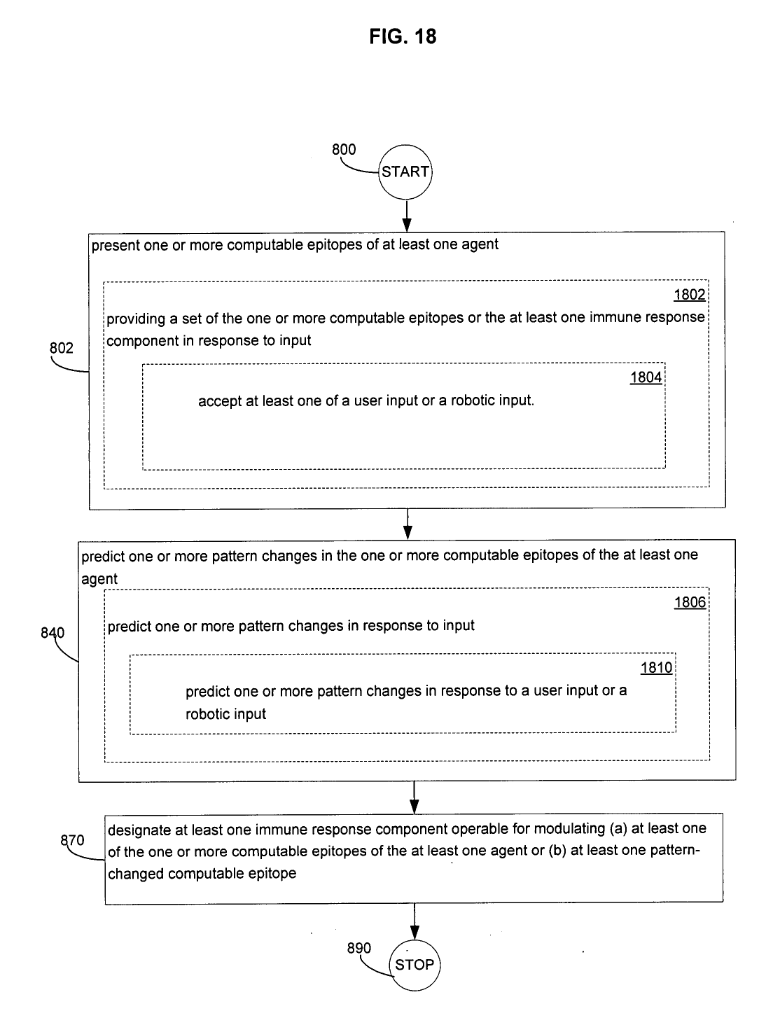

With reference to the figures, and with reference now to FIG. 18, depicted is a high-level logic flowchart depicting alternate implementations of the high-level logic flowchart of FIG. 8. Illustrated is that in various alternate implementations method step 802 may include method step 1802. Method step 1802 illustrates providing a set of the one or more computable epitopes or the at least one immune response component in response to input. In some implementations method step 1802 may include method step 1804. Method step 1804 depicts accepting at least one of a user input or a robotic input. Illustrated is that in various alternate implementations method step 840 may include method step 1806. Method step 1806 depicts predicting one or more pattern changes in response to input. In some implementations, method step 1806 may include method step 1810. Method step 1810 depicts predicting one or more pattern changes in response to a user input or a robotic input.

C. Variation(s), and/or Implementation(s)

Those having skill in the art will recognize that the present application teaches modifications of the devices, structures, and/or processes within the spirit of the teaching herein. For example, in one aspect, the immune response components may be formulated to cross the blood-brain barrier which is known to exclude mostly hydrophilic compounds, as well as to discriminate against transport of high molecular weight ones. For example, an antibody fragment may be encased in a lipid vesicle. In another example, the antibody or a portion of the antibody may be tagged onto a carrier protein or molecule. In another example, an antibody or other immune response component may be split into one or more complementary fragments, each fragment encased by a lipid vesicle, and each fragment functional only on binding its complementary fragment. Once the blood-brain barrier has been crossed the lipid vesicle may be dissolved to release the antibody fragments which reunite with their complementary counterparts and may form a fully functional antibody or other immune response component. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the immune response components may be made in large format. The method lends itself to both small format or personalized care applications and large-scale or large format applications. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the method may be used to designate immune response components for any diseases or disorders. The application of this method is not limited to diseases where antigenic shift or drift keeps the immune system ‘guessing’ or causing it to be effectively slow-to-respond. Although, influenza or HIV-1 are likely viral-disease-agent candidates for application of this method, treatment of other diseases, disorders and/or conditions will likely benefit from this methodology. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, real-time evaluation may be provided of the antigenic changes by including a portable PCR machine which samples the environment for (sub-) strains of pathogens locally present. The information may be sent remotely to another location or to a portable material-administering device utilized by the affected person, for example, a drip-patch device with a remote sensor, resulting in the activation of the necessary immune response components and thereby providing adequate protection. As the evaluation possibly changes in time, the portable administering device may be controlled to change the dosage or type of immune response component delivered. Such a portable administering device operably coupled to a portable PCR machine or a functionally similar system has a wide variety of applications, for example, including, but not limited to, when medical personnel visit an area in which one or more diseases may be endemic, and/or when military personnel visit hostile territory in which unknown pathogens may be present. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, an individual may use an administering device containing the immune response components that is preprogrammed to provide the user the necessary immune response-mediated protection over an interval of time, and/or to anticipate pattern changes in the epitopes of the agent 100. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, RNA blockers, and/or single- or double-stranded RNA interference technology may be used to down-regulate expression of genes or to reduce concentrations of their expression products or resulting components of the immune system in conjunction with the method. Other modifications of the subject matter herein will be appreciated by one of skill in the art in light of the teachings herein.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency vs. operational convenience tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary substantially.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other extensively-integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter subject matter described herein applies equally regardless of the particular type of signal-bearing media used to actually carry out the distribution. Examples of a signal-bearing media include, but are not limited to, the following: recordable type media such as floppy disks, hard disk drives, DVD/CD-ROMs, digital tape, and computer memory devices of various types; and data transmission-type media such as digital and analog communication links using TDM or IP-based communication links (e.g., packetized data links).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application-specific integrated circuit, electrical circuitry forming a general-purpose computing device configured by a computer program (e.g., a general-purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into data-processing systems. That is, at least a portion of the devices and/or processes described herein can be integrated into a data-processing system via a reasonable amount of experimentation. Those having skill in the art will recognize that a typical data-processing system generally includes one or more of a system unit housing, a display device, a memory such as volatile and/or non-volatile memory, processors such as microprocessors and digital signal processors, computational entities such as operating systems, drivers, (e.g., graphical) user interfaces, and applications programs, one or more interaction devices, such as a touch pad or screen, and/or control systems including feedback loops and control motors (e.g., feedback for sensing position and/or velocity; control motors for moving and/or adjusting components such as valves and/or quantities). A typical data-processing system may be implemented utilizing any suitable commercially available components, such as those typically found in digital computing/communication and/or network computing/communication systems.

All of the referenced U.S. patents, U.S. patent application publications, U.S. patent applications, foreign patents, foreign patent applications, and/or non-patent publications referred to in this specification and/or listed in any Application Data Sheet, are incorporated herein by reference, in their entireties.

The herein described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

1. A method, comprising:
presenting one or more computable epitopes of at least one agent;
predicting one or more pattern changes in the one or more computable epitopes of the at least one agent; and
designating at least one immune response component operable for modulating (a) at least one of the one or more computable epitopes of the at least one agent or (b) at least one pattern-changed computable epitope.

2. The method of claim 1, wherein the presenting one or more computable epitopes of at least one agent further comprises:
presenting at least a portion of at least one of a polyglycopeptide, a nucleic acid, or a toxin.

3. The method of claim 1, wherein the presenting one or more computable epitopes of at least one agent further comprises:
presenting at least a part of at least one of a nucleotide, a carbohydrate, a lipid, a polysaccharide, a lipopolysaccharide, a glycolipid, a polyglycopeptide, or a glycoprotein.

4. The method of claim 1, wherein the presenting one or more computable epitopes of at least one agent further comprises:
presenting one or more computable epitopes having at least nine nucleotides.

5. The method of claim 1, wherein the presenting one or more computable epitopes of at least one agent further comprises:
presenting one or more computable epitopes having at least one sugar moiety.

6. The method of claim 1, wherein the presenting one or more computable epitopes of at least one agent further comprises:
presenting one or more substantially non-linear computable epitopes.

7. The method of claim 1, wherein the presenting one or more computable epitopes of at least one agent further comprises:
providing a set of one or more computable epitopes of the at least one agent wherein the set includes at least one computable epitope with up to substantially 80% amino acid sequence match with at least one host.

8. The method of claim 1, wherein the presenting one or more computable epitopes of at least one agent further comprises:
providing a set of one or more computable epitopes of the at least one agent wherein the set includes at least one computable epitope with up to substantially 70% amino acid sequence match with at least one host.

9. The method of claim 1, wherein the presenting one or more computable epitopes of at least one agent further comprises:
providing a set of one or more computable epitopes of the at least one agent wherein the set includes at least one computable epitope with up to substantially 60% amino acid sequence match with at least one host.

10. The method of claim 1, wherein the presenting one or more computable epitopes of at least one agent further comprises:
providing a set of one or more computable epitopes of the at least one agent wherein the set includes at least one computable epitope having between substantially 0% to substantially 80% sequence match with at least one host.

11. The method of claim 1, wherein the presenting one or more computable epitopes of at least one agent further comprises:
providing a set of one or more computable epitopes of the at least one agent wherein the set includes at least one computable epitope having a likely sequence match with at least one host.

12. (canceled)

13. The method of claim 1, wherein the presenting one or more computable epitopes of at least one agent further comprises:
providing a set of one or more computable epitopes of the at least one agent wherein the set includes at least one computable epitope having at least 87% sequence match with at least one host.

14. The method of claim 1, wherein the presenting one or more computable epitopes of at least one agent further comprises:
providing a set of one or more computable epitopes of the at least one agent wherein the set includes at least one computable epitope having a substantially similar functional sequence match with at least one host.

15. The method of claim 1, wherein the presenting one or more computable epitopes of at least one agent further comprises:
providing a set of one or more computable epitopes of the at least one agent wherein the set includes at least one computable epitope having a substantially similar structural match with at least one host.

16. The method of claim 1, wherein the predicting one or more pattern changes in the one or more computable epitopes of the at least one agent further comprises:
predicting one or more changes in one or more sugar moieties of the at least one agent.

17. The method of claim 1, wherein the designating at least one immune response component further comprises:
designating at least a part of a humanized antibody.

18. The method of claim 1, wherein the designating at least one immune response component further comprises:
designating at least one modulator of at least a part of a humanized antibody.

19. The method of claim 1, wherein the designating at least one immune response component further comprises:
designating an immune response component directed to at least one secreted protein.

20. The method of claim 1, wherein the designating at least one immune response component further comprises:
designating at least one modulator of (a) an epitopic shift or (b) an epitopic drift predicted in the at least one agent.

21. The method of claim 20, wherein the designating at least one modulator of (a) an epitopic shift or (b) an epitopic drift predicted in the at least one agent further comprises:
designating at least one suppressor of mutagenesis of the at least one agent.

22. The method of claim 20, wherein the designating at least one modulator of (a) an epitopic shift or (b) an epitopic drift predicted in the at least one agent further comprises:
designating at least one interfering nucleic acid.

23. The method of claim 22, wherein the at least one interfering nucleic acid further comprises:
one or more ribonucleotides.

24. The method of claim 22, wherein the at least one interfering nucleic acid further comprises:
one or more of a deoxynucleotide, a chemically synthesized nucleotide, a nucleotide analog, a nucleotide not naturally occurring, a nucleotide not found in natural PNA, or a nucleotide not found in natural DNA of an untreated agent.

25. A system, comprising:
circuitry for presenting one or more computable epitopes of at least one agent;
circuitry for predicting one or more pattern changes in the one or more computable epitopes of the at least one agent; and
circuitry for designating at least one immune response component operable for modulating (a) at least one of the one or more computable epitopes of the at least one agent or (b) at least one pattern-changed computable epitope.

26. The system as in claim 25, wherein the circuitry for presenting one or more computable epitopes of at least one agent further comprises:
circuitry for presenting at least a portion of at least one of a polyglycopeptide, a nucleic acid, or a toxin.

27. The system as in claim 25, wherein the circuitry for presenting one or more computable epitopes of at least one agent further comprises:
circuitry for presenting at least a part of at least one of a nucleotide, a carbohydrate, a lipid, a polysaccharide, a lipopolysaccharide, a glycolipid, a polyglycopeptide, or a glycoprotein.

28. The system as in claim 25, wherein the circuitry for presenting one or more computable epitopes of at least one agent further comprises:
circuitry for presenting one or more computable epitopes having at least nine nucleotides.

29. The system as in claim 25, wherein the circuitry for presenting one or more computable epitopes of at least one agent further comprises:
circuitry for presenting one or more computable epitopes having at least one sugar moiety.

30. The system as in claim 25, wherein the circuitry for presenting one or more computable epitopes of at least one agent further comprises:
circuitry for presenting one or more substantially non-linear computable epitopes.

31. The system as in claim 25, wherein the circuitry for presenting one or more computable epitopes of at least one agent further comprises:
circuitry for providing a set of one or more computable epitopes of the at least one agent wherein the set includes at least one computable epitope with up to about 80% amino acid sequence match with at least one host.

32. The system as in claim 25, wherein the circuitry for presenting one or more computable epitopes of at least one agent further comprises:
circuitry for providing a set of one or more computable epitopes of the at least one agent wherein the set includes at least one computable epitope with up to about 70% amino acid sequence match with at least one host.

33. The system as in claim 25, wherein the circuitry for presenting one or more computable epitopes of at least one agent further comprises:
circuitry for providing a set of one or more computable epitopes of the at least one agent wherein the set includes at least one computable epitope with up to about 60% amino acid sequence match with at least one host.

34. The system as in claim 25, wherein the circuitry for presenting one or more computable epitopes of at least one agent further comprises:
circuitry for providing a set of one or more computable epitopes of the at least one agent wherein the set includes at least one computable epitope having between substantially 0% to substantially 80% sequence match with at least one host.

35. The system as in claim 25, wherein the circuitry for presenting one or more computable epitopes of at least one agent further comprises:
circuitry for providing a set of one or more computable epitopes of the at least one agent wherein the set includes at least one computable epitope having a likely sequence match with at least one host.

36. (canceled)

37. The system as in claim 25, wherein the circuitry for presenting one or more computable epitopes of at least one agent further comprises:
circuitry for providing a set of one or more computable epitopes of the at least one agent wherein the set includes at least one computable epitope having at least 87% sequence match with at least one host.

38. The system as in claim 25, wherein the circuitry for presenting one or more computable epitopes of at least one agent further comprises:
circuitry for providing a set of one or more computable epitopes of the at least one agent wherein the set includes at least one computable epitope having a substantially similar functional sequence match with at least one host.

39. The system as in claim 25, wherein the circuitry for presenting one or more computable epitopes of at least one agent further comprises:
circuitry for providing a set of one or more computable epitopes of the at least one agent wherein the set includes at least one computable epitope having a substantially similar structural match with at least one host.

40. The system as in claim 25, wherein the circuitry for predicting one or more pattern changes in the one or more computable epitopes of the at least one agent further comprises:
circuitry for predicting one or more changes in one or more sugar moieties of the at least one agent.

41. The system as in claim 25, wherein the circuitry for designating at least one immune response component further comprises:
circuitry for designating at least a part of at least one a humanized antibody.

42. The system as in claim 25, wherein the circuitry for designating at least one immune response component further comprises:
circuitry for designating at least one modulator of at least a part of at least one humanized antibody.

43. The system as in claim 25, wherein the circuitry for designating at least one immune response component further comprises:
circuitry for designating an immune response component directed to at least one secreted protein.

44. The system as in claim 25, wherein the circuitry for designating at least one immune response component further comprises:
circuitry for designating at least one modulator of (a) an epitopic shift or (b) an epitopic drift predicted in the at least one agent.

45. The system as in claim 44, wherein the circuitry for designating at least one modulator of (a) an epitopic shift or (b) an epitopic drift predicted in the at least one agent further comprises:
circuitry for designating at least one suppressor of mutagenesis of the at least one agent.

46. The system as in claim 44, wherein the circuitry for designating at least one modulator of (a) an epitopic shift or (b) an epitopic drift predicted in the at least one agent further comprises:
circuitry for designating at least one interfering nucleic acid.

47. The system as in claim 46, wherein the circuitry for designating at least one interfering nucleic acid further comprises:
circuitry for designating one or more ribonucleotides.

48. The system as in claim 46, wherein the circuitry for designating at least one interfering nucleic acid further comprises:
one or more of a deoxynucleotide, a chemically synthesized nucleotide, a nucleotide analog, a nucleotide not naturally occurring, a nucleotide not found in natural RNA, or a nucleotide not found in natural DNA of an untreated agent.

49. A system, comprising:
means for presenting one or more computable epitopes of at least one agent;
means for predicting one or more pattern changes in the one or more computable epitopes of the at least one agent; and
means for designating at least one immune response component operable for modulating at least one pattern-changed computable epitope.

50. A system, comprising:
a computer readable medium including, but not limited to, a computer program for use with a computer system and wherein the computer program includes a plurality of instructions including
one or more instructions for presenting one or more computable epitopes of at least one agent,
one or more instructions for predicting one or more pattern changes in the one or more computable epitopes of the at least one agent, and
one or more instructions for designating at least one immune response component operable for modulating at least one pattern-changed computable epitope.

51. A program product, comprising:
at least one signal-bearing medium including
one or more instructions for presenting one or more computable epitopes of at least one agent,
one or more instructions for predicting one or more pattern changes in the one or more computable epitopes of the at least one agent, and
one or more instructions for designating at least one immune response component operable for modulating at least one pattern-changed computable epitope.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)
